(19) United States
(12) Patent Application Publication
(10) Pub. No.: US 2004/0127686 A1
(43) Pub. Date: Jul. 1, 2004

(54) Methods and compositions relating to muscle specific sarcomeric calcineurin-binding proteins (calsarcins)

(75) Inventors: Eric Olson, Dallas, TX (US); Norbert Frey, Dallas, TX (US)

Correspondence Address: Steven L. Highlander, Esq., FULBRIGHT & JAWORSKI L.L.P., Suite 2400, 600 Congress Avenue, Austin, TX, 78701, US

(73) Assignee: Board of Regents, The University of Texas System

(21) Appl. No.: 10/759,624

(22) Filed: Jan. 16, 2004

Related U.S. Application Data
(62) Division of application No. 10/045,594, filed on Nov. 7, 2001.
(60) Provisional application No. 60/246,629, filed on Nov. 7, 2000.

(51) Int. Cl. C07K 14/47; A01K 67/00; C07H 21/04

(52) U.S. Cl. 530/350; 435/69.1; 435/320.1; 435/325; 536/23.5

(57) ABSTRACT

The present invention relates to the polypeptides known as calcineurin associated protein (calsarcin). Calsarcins-1, -2, and -3 bind to calcineurin, telethonin, and α-actinin, which provides a link between these molecules and the sarcomere. Sarcomeric dysfunction ultimately leads to activation of calcineurin and consequent hypertrophic cardiomyopathy. Thus, methods utilizing calsarcin as it regards these medical conditions are herein provided and include screening for peptides which interact with calsarcin, screening for modulators of calsarcin binding to calcineurin or α-actinin, methods to modulate calcineurin activity, methods to inhibit calcineurin activation of gene transcription and methods for treating cardiac hypertrophy, heart failure and Type II diabetes.

The present application claims priority to co-pending U.S. Provisional Patent Application Serial No. 60/246,629 filed on Nov. 7^{th}, 2000. The entire text of the above-referenced disclosure is specifically incorporated herein by reference without disclaimer. The government may own rights in the present invention pursuant to grant number HL53351-06 from the National Institutes of Health.

### BACKGROUND OF THE INVENTION

1. Field of the Invention

The present invention relates generally to the fields of cell biology and molecular biology. Particularly, it concerns the regulation of activity of calcineurin through a calcineurin-associated sarcomeric protein (calsarcin). More particularly, it concerns the regulation of activity of calcineurin through CALSARCIN-1, which also interacts with the sarcomere-related α-actinin.

2. Description of Related Art

Calcineurin is a serine/threonine protein phosphatase that plays a pivotal role in developmental and homeostatic regulation of a wide variety of cell types (Klee et al, 1998; Crabtree, 1999). The interaction of calcineurin with transcription factors of the NFAT family following activation of the T cell receptor in leukocytes provides the best characterized example of how calcineurin regulates gene expression (Rao et al, 1997). Changes in intracellular calcium promote binding of Ca²⁺/calmodulin to the catalytic subunit of calcineurin (CnA), thereby displacing an autoinhibitory region and allowing access of protein substrates to the catalytic domain. Dephosphorylation of NFAT by activated calcineurin promotes its translocation from the cytoplasm to the nucleus, where NFAT binds DNA cooperatively with an AP1 heterodimer to activate transcription of genes encoding cytokines, such as IL-2. This basic model of NFAT activation has been shown to transduce Ca²⁺ signals via calcineurin in many cell types and to control transcription of diverse sets of target genes unique to each cellular environment (Timmerman et al, 1996). In each case, NFAT acts cooperatively with other transcription factors that include proteins of the API (Rao et al, 1997), cMAF (Ho et al, 1996), GATA (Mesaeli et al, 1999; Molkentin et al, 1998; Musaro et al, 1999), or MEF2 (Chin et al, 1998; Liu et al, 1997; Mao et al, 1999; Mao and Wiedmann, 1999) families. In addition to T cell activation, cellular responses controlled by calcineurin signaling include synaptic plasticity (Mao et al, 1999; Graef et al, 1999; Zhuo et al, 1999) and apoptosis (Wang et al, 1999; Youn et al, 1999).

Recent studies of calcineurin signaling in striated myocytes of heart and skeletal muscle have expanded the scope of important physiological and pathological events controlled by this ubiquitously expressed protein. Forced expression of a constitutively active form of calcineurin in hearts of transgenic mice promotes cardiac hypertrophy that progresses to dilated cardiomyopathy, heart failure, and death, in a manner that recapitulates features of human disease (Molkentin et al, 1998, herein incorporated by reference). Moreover, hypertrophy and heart failure in these animals, and in certain other animal models of cardiomyopathy, are prevented by administration of the calcineurin antagonist drugs cyclosporin A or FK-506 (Sussman et al, 1998). In skeletal muscles, calcineurin signaling is implicated both in hypertrophic growth stimulated by insulin-like growth factor-1 (Musaro et al, 1999; Semsarian et al, 1999), and in the control of specialized programs of gene expression that establish distinctive myofiber subtypes (Chin et al, 1998; Dunn et al, 1999). These observations have stimulated interest in the therapeutic potential of modifying calcineurin activity selectively in muscle cells while avoiding unwanted consequences of altered calcineurin signaling in other cell types (Sigal et al, 1991).

The activity of calcineurin in mammalian cells can be modulated by interactions with other proteins. These include not only immunophilins that are the targets of the immunosuppressant drugs cyclosporin A and FK-506, but two unrelated proteins (AKAP79 and cabin-1/cain) that were identified recently. AKAP79 binds calcineurin in conjunction with protein kinase C and protein kinase A, serving as a scaffold for assembly of a large hetero-oligomeric signaling complex (Kashishian et al, 1998). Cabin-1/cain binds both calcineurin and the transcription factor MEF2 (Sun et al, 1998; Lai et al, 1998). As a consequence of cabin-1 overexpression, calcineurin activity is inhibited and MEF2 is sequestered in an inactive state. Another calcineurin-binding protein is Rex1p (YKL159c) of Saccharomyces cerevisiae. A preliminary report noted that this small 24 kDa protein inhibits calcineurin signaling when overexpressed in yeast (Kingsbury and Cunningham, 1998).

In muscle cells, the actin filaments of the cytoskeleton are stably anchored at the Z-disk of the sarcomere, and furthermore are required for the transmission of mechanical strain along the length of the muscle through the serially ordered sarcomeres. The Z-disk consists of the anti-parallel dimeric actin-binding protein α-actinin (Luther, 1991). For a given actin filament, there is overlap of four filaments from the opposite sarcomere which results in the formation of a square grid cross-connected in a zig-zag pattern by the α-actinin-composed Z filaments. The periodicity of α-actinin in this grid is between 15 and 20 nm (Luther, 1991; Schroeter et al, 1996) and, although the number of α-actinin cross-links is variable, the total number is highly regulated in a given muscle fiber (Squire, 1981; Vigoreaux, 1994).

Sarcomeric α-actinin, (s-α-actinin) and the α-actinin present in non-muscle cells (non-s-α-actinin) are encoded by two different genes. Furthermore, isoforms of s-α-actinin are produced likely through alternative splicing schemes (Baron et al, 1987; de Arruda et al, 1990; Beggs et al, 1992; Parr et al, 1992). Actin binding of the non-s-α-actinin form is Ca²⁺-sensitive, whereas actin binding of the s-α-actinin form is Ca²⁺-insensitive (Burridge and Feramisco, 1980; Duhaiman and Banburg, 1984; Bennett et al, 1984; Landon et al, 1985).

Drosophila α-actinin gene mutants are lethal, although the flies are able to survive beyond embryogenesis with detectable muscle dysfunction present at the hatching stage (Fyrberg et al, 1998). In larval development, the mutation manifests through noticeable muscle degeneration which progressively limits mobility, and ultimately leads to death. Microscopic evaluation of mutant muscle fibers indicates that in as early as one-day old larvae, myofibrils are significantly perturbed with similar cellular pathologies to human nemaline myopathies.

Telethonin is sarcomeric protein of heart and skeletal muscle encoded by the gene involved in limb-girdle muscular dystrophy. Muscular dystrophy (MD) refers to a group of genetic diseases characterized by progressive weakness and degeneration of the skeletal or voluntary muscles which control movement. The muscles of the heart and some other involuntary muscles are also affected in some forms of MD, and a few forms involve other organs as well. The major forms of MD include myotonic, Duchenne, Becker, limb-girdle, facioscapulohumeral, congenital, oculopharyngeal, distal and Emery-Dreifuss. Duchenne is the most common form of MD affecting children, and myotonic MD is the most common form affecting adults. MD can affect people of all ages. Although some forms first become apparent in infancy or childhood, others may not appear until middle age or later. There is no known cure for muscular dystrophy therefore, gene therapies with calsarcins may prove valuable.

Previous studies (Sussman et al, 1998; Shimoyama et al, 1999; Hill et al, 2000; Lim et al, 2000a; Lim et al, 2000b; Taigen et al, 2000) have demonstrated that sarcomeric dysfunction with resulting alterations in calcium handling results in activation of calcineurin and consequent hypertrophic cardiomyopathy. A link between calcineurin and the sarcomere, such as with a calcineurin associated protein or peptide, provides a therapeutic target. Identification of new, more suitable candidates having the ability to modulate calcineurin function in cardiac tissue is an important goal of current research efforts.

Since the time of the initial discovery of the central role of calcineurin in cardiac hypertrophy and heart failure (Molkentin et al, 1998), there have been numerous follow-up studies that have confirmed the importance of this signaling pathway in hypertrophic growth of the heart in response to diverse intrinsic and extrinsic signals (reviewed in Olson and Molkentin, 1999; Izumo and Aoki, 1998). Inhibition or activation of this pathway in the heart can have profound consequences on cardiac cell growth and has important therapeutic implications. However, the importance of calcineurin for T-cell activation results in immunosuppression when calcineurin is globally inhibited in the entire organism. Thus, the identification of cardiac-specific calcineurin-binding proteins could allow for possible tissue-specific means of altering calcineurin activity in the heart through targeting the protein to specific subcellular sites or through modification of the cardiac-specific target proteins.

### SUMMARY OF THE INVENTION

The invention employs a novel protein calsarcin, which links calcineurin to α-actinin within the sarcomere. Using dominant negative mutant versions of calsarcin as “decoys,” calcineurin can be misdirected within a cardiac myocyte to an inappropriate intracellular location, thereby disrupting calcineurin hypertrophic signaling. These decoys, which, in specific embodiment, could contain portions of calsarcin that associate with calcineurin but not with α-actinin, could be expressed in cardiac myocytes in vitro by adenovirus-mediated gene delivery

In an embodiment of the present invention, there is an isolated and purified polypeptide comprising SEQ ID NO:2, SEQ ID NO:4, SEQ ID NO:6, SEQ ID NO:8, SEQ ID NO:10, or SEQ ID NO:12.

In an additional embodiment of the present invention, there is an isolated and purified nucleic acid comprising a nucleic acid segment encoding SEQ ID NO:1, SEQ ID NO:3, SEQ ID NO:5, SEQ ID NO:7, SEQ ID NO:9, SEQ ID NO:11 In a specific embodiment, a nucleic acid segment further comprises a promoter active in eukaryotic cells. In another specific embodiment, a nucleic acid further comprises a recombinant vector.

In another embodiment of the present invention there is an isolated and purified nucleic acid segment, wherein said nucleic acid segment encodes a fusion polypeptide comprising SEQ ID NO:2. In another embodiment of the present invention there is an isolated and purified nucleic acid segment, wherein said nucleic acid segment encodes a fusion polypeptide comprising SEQ ID NO:4, 6, 8, 10, or 12.

In an additional embodiment of the present invention, there is a knockout non-human animal comprising a defective allele of a nucleic acid encoding calsarcin. In a specific embodiment, the animal further comprises two defective alleles of a nucleic acid encoding calsarcin. In an additional specific embodiment, the animal is a mouse.

In an additional embodiment of the present invention, there is a transgenic non-human animal comprising an expression cassette, wherein said cassette comprises a nucleic acid encoding a calsarcin polypeptide under the control of a promoter active in eukaryotic cells. In specific embodiments, the promoter is constitutive, tissue specific, or inducible. In another specific embodiment the animal is a mouse.

In another embodiment of the present invention, there is a monoclonal antibody that binds immunologically to a polypeptide comprising SEQ ID NO:2, or an antigenic fragment thereof. In another embodiment of the present invention, there is a monoclonal antibody that binds immunologically to a polypeptide comprising SEQ ID NO:4, 6, 8, 10, or 12, or an antigenic fragment thereof.

In an additional embodiment of the present invention, there is polyclonal antisera, antibodies of which bind immunologically to a polypeptide comprising SEQ ID NO:2, or an antigenic fragment thereof. In an additional embodiment of the present invention, there is polyclonal antisera, antibodies of which bind immunologically to a polypeptide comprising SEQ ID NO:4, 6, 8, 10, or 12, or an antigenic fragment thereof.

In an additional embodiment of the present invention, there is a method of modulating calcineurin activity in an animal comprising the step of administering to said organism a calsarcin polypeptide, or a calcineurin-binding fragment thereof.

In a further embodiment of the present invention, there is a method of modulating calcineurin activity in an animal comprising the step of administering to said organism a dominant-negative form of a calsarcin polypeptide, or a calcineurin-binding fragment thereof.

In an additional embodiment of the present invention, there is a method of modulating calcineurin activity in an animal comprising the step of administering to said animal a nucleic acid which encodes a calsarcin polypeptide, or a calcineurin-binding fragment thereof, said nucleic acid under the control of a promoter operable in cells of said animal. In specific embodiments, the promoter is a constitutive promoter or a muscle-specific promoter. In another specific embodiment, the muscle-specific promoter is myosin light chain-2 promoter, a actin promoter, troponin 1 promoter, Na⁺/Ca²⁺ exchanger promoter, dystrophin promoter, creatine kinase promoter, α7 integrin promoter, brain natriuretic peptide promoter, α B-crystallin/small heat shock protein promoter, a myosin heavy chain promoter or atrial natriuretic factor promoter. In another specific embodiment, the nucleic acid comprises a viral vector.

In another embodiment of the present invention, there is a method of screening for a peptide which interacts with calsarcin comprising the steps of introducing into a cell a first nucleic acid comprising a DNA segment encoding a test peptide, wherein said test peptide is fused to a DNA binding domain; and a second nucleic acid comprising a DNA segment encoding at least a part of calsarcin, wherein said at least part of calsarcin is fused to a DNA activation domain; and assaying for an interaction between said test peptide and said at least part of calsarcin by assaying for an interaction between said DNA binding domain and said DNA activation domain. In a specific embodiment, a DNA binding domain and a DNA activation domain are selected from the group consisting of GAL4 and LexA.

In an additional embodiment of the present invention, there is a method of screening for a modulator of calsarcin binding to α-actinin comprising providing a calsarcin and α-actinin; admixing the calsarcin and α-actinin in the presence of a candidate modulator; measuring calsarcin/α-actinin binding; and comparing the binding in step (c) with the binding of calsarcin and α-actinin in the absence of said candidate modulator, whereby a difference in the binding of calsarcin and α-actinin in the presence of said candidate modulator, as compared to binding in the absence of said candidate modulator, identifies said candidate modulator as a modulator of calsarcin binding to α-actinin. In a specific embodiment, calsarcin and α-actinin are part of a cell free system. In another specific embodiment, calsarcin and α-actinin are located within an intact cell. In an additional specific embodiment, the cell is a myocyte. In a further specific embodiment, the cell is a H9C2 cell, a C2C12 cell, a 3T3 cell, a 293 cell, a neonatal cardiomyocyte cell, an adult cardiomyocyte or a myotube cell. In an additional specific embodiment, the intact cell is located in an animal. In a further specific embodiment the modulator increases or decreases calsarcin binding to α-actinin. In another specific embodiment, either or both calsarcin and α-actinin are labeled. In another specific embodiment, both calsarcin and α-actinin are labeled, one with a quenchable label and the other with a quenching agent. In an additional specific embodiment, both calsarcin and α-actinin are labeled, but said labels are not detectable unless brought into proximity of each other. In a further specific embodiment, the measuring comprises immunologic detection of calsarcin, α-actinin or both. In another specific embodiment, the method further comprises measuring binding of calsarcin and α-actinin in the absence of a modulator.

In another embodiment of the present invention, there is a method of screening for a modulator of calsarcin binding to calcineurin comprising providing a calsarcin and calcineurin; admixing the calsarcin and calcineurin in the presence of a candidate modulator; measuring calsarcin/calcineurin binding; and comparing the binding in step (c) with the binding of calsarcin and calcineurin in the absence of said candidate modulator, whereby a difference in the binding of calsarcin and calcineurin in the presence of said candidate modulator, as compared to binding in the absence of said candidate modulator, identifies said candidate modulator as a modulator of calsarcin binding to calcineurin. In a specific embodiment, the calsarcin and calcineurin are part of a cell free system. In another specific embodiment, the calsarcin and calcineurin are located within an intact cell. In an additional specific embodiment, the cell is a myocyte. In a further specific embodiment, the cell is a H9C2 cell, a C2C12 cell, a 3T3 cell, a 293 cell, a neonatal cardiomyocyte cell, an adult cardiomyocyte or a myotube cell. In a further specific embodiment, the intact cell is located in an animal. In another specific embodiment, the modulator increases or decreases calsarcin binding to calcineurin. In a further specific embodiment, both calsarcin and calcineurin are labeled. In another specific embodiment, both calsarcin and calcineurin are labeled, one with a quenchable label and the other with a quenching agent. In an additional specific embodiment, both calsarcin and calcineurin are labeled, but said labels are not detectable unless brought into proximity of each other. In another specific embodiment, the measuring comprises immunologic detection of calsarcin, calcineurin or both. In an additional embodiment, the method further comprises measuring binding of calsarcin and calcimeurin in the absence of a modulator.

In another embodiment of the present invention, there is a method of screening for a modulator of calsarcin binding to telethonin comprising providing a calsarcin and telethonin; admixing the calsarcin and telethonin in the presence of a candidate modulator; measuring calsarcin/telethonin binding; and comparing the binding in step (c) with the binding of calsarcin and telethonin in the absence of said candidate modulator, whereby a difference in the binding of calsarcin and telethonin in the presence of said candidate modulator, as compared to binding in the absence of said candidate modulator, identifies said candidate modulator as a modulator of calsarcin binding to telethonin. In a specific embodiment, the calsarcin and telethonin are part of a cell free system. In another specific embodiment, the calsarcin and telethonin are located within an intact cell. In an additional specific embodiment, the cell is a myocyte. In a further specific embodiment, the cell is a H9C2 cell, a C2C12 cell, a 3T3 cell, a 293 cell, a neonatal cardiomyocyte cell, an adult cardiomyocyte or a myotube cell. In a further specific embodiment, the intact cell is located in an animal. In another specific embodiment, the modulator increases or decreases calsarcin binding to telethonin. In a further specific embodiment, both calsarcin and telethonin are labeled. In another specific embodiment, both calsarcin and telethonin are labeled, one with a quenchable label and the other with a quenching agent. In an additional specific embodiment, both calsarcin and telethonin are labeled, but said labels are not detectable unless brought into proximity of each other. In another specific embodiment, the measuring comprises immunologic detection of calsarcin, telethonin or both. In an additional embodiment, the method further comprises measuring binding of calsarcin and telethonin in the absence of a modulator.

In another embodiment of the present invention, there is a method of treating cardiac hypertrophy, heart failure or Type II diabetes comprising the step of administering to an animal suffering therefrom a calsarcin polypeptide, or a calcineurin-binding fragment thereof, wherein said calsarcin polypeptide or fragment thereof inhibits calcineurin activity.

In an additional embodiment of the present invention, there is a method of treating cardiac hypertrophy, heart failure or Type II diabetes comprising the step of administering to an animal suffering therefrom a nucleic acid encoding a calsarcin polypeptide or a calcineurin binding fragment thereof, under the control of a promoter active in cardiac tissue, wherein expression of said calsarcin polypeptide or fragment thereof inhibits calcineurin activity. Also, an inhibitor may be any molecule that interferes with calcineurin-calsarcin, or α-actinin interactions. In a specific embodiment, the polypeptide is a dominant negative form of calsarcin. In another specific emboidment, the method further comprises treating said animal with a compound selected from the group consisting of an ionotrope, a beta blocker, an antiarrhythmic, a diuretic, a vasodilator, a hormone antagonist, an endothelin antagonist, an angiotensin type 2 antagonist and a cytokine inhibitor/blocker. In an additional specific embodiment, the promoter is a constitutive promoter or an inducible promoter.

In an additional embodiment of the present invention, there is a method of inhibiting calcineurin activation of gene transcription in a cell comprising providing to said cell a fusion protein comprising calsarcin, or a calcineurin-binding fragment thereof, fused to a targeting peptide that localizes said fusion protein to a subcellular region other than a subcellular region of normal function. In a specific embodiment, a targeting peptide comprises a geranylgeranyl group, a nuclear localization signal, a myristilation signal, and an endoplasmic reticulum signal peptide. In another specific embodiment, a cell is located in an animal. In a further specific embodiment, the animal is a human. In an additional specific embodiment the method further comprises treating said animal with a compound selected from the group consisting of an ionotrope, a beta blocker, an antiarrhythmic, a diuretic, a vasodilator, a hormone antagonist, an endothelin antagonist, an angiotensin type 2 antagonist and a cytokine inhibitor/blocker.

In another embodiment of the present invention, there is a method of identifying a peptide that binds calsarcin comprising the steps of attaching a calsarcin polypeptide, or a fragment thereof, to a support; exposing said calsarcin polypeptide or fragment to a candidate peptide; and assaying for binding of said candidate peptide to said calsarcin polypeptide or fragment thereof. In a specific embodiment the support is selected from the group consisting of nitrocellulose, a column, or a gel.

In an additional embodiment of the present invention, there is a method of screening for a candidate substance for anti-cardiomyopic hypertrophy activity or anti-heart failure activity comprising the steps of providing a cell lacking a functional calsarcin polypeptide; contacting said cell with said candidate substance; and determining the effect of said candidate substance on said cell. In a specific embodiment, the cell is a muscle cell. In another specific embodiment, the cell has a mutation in a regulatory region of calsarcin. In a further specific embodiment the mutation is a deletion mutation, an insertion mutation, or a point mutation. In a specific embodiment, the cell has a mutation in the coding region of calsarcin. In another specific embodiment, the mutation is a deletion mutation, an insertion mutation, a frameshift mutation, a nonsense mutation, a missense mutation or a splicing mutation. In further specific embodiments, the cell is contacted in vitro or in vivo. In an additional specific embodiment, the cell is located in a non-human transgenic animal

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E—Predicted amino acid sequences of human and mouse calsarcin-1 and calsarcin-2. The deduced amino acid sequences of human calsarcin-1 (FIG. 1A), mouse calsarcin-1 (FIG. 1B), human calsarcin-2 (FIG. 1C) and mouse calsarcin-2 (FIG. 1D) are shown, along with an amino acid alignment of the mouse proteins (FIG. 1E).

FIGS. 2A-D—Nucleotide sequences for human calsarcin-1 (FIG. 2A), mouse calsarcin-1 (FIG. 2B), human calsarcin-2 (FIG. 2C) and mouse calsarcin-2 (FIG. 2D).

FIG. 3—Northern blot analysis of calsarcin-1 and calsarcin-2 in adult human and mouse tissues. Calsarcin transcripts were detected by Northern analysis of the indicated human and mouse tissues. Calsarcin-1 mRNA is predominantly detected in heart and skeletal muscle, whereas the calsarcin-2 transcript was detected in skeletal muscle of both species.

FIGS. 4A-E—Developmental expression of calsarcin-1 and -2. FIG. 4A: Calsarcin-1 and -2 transcripts were detected by radioactive in situ hybridization of mouse embryo sagittal sections at the embryonic time points indicated above each set of panels (b, brain; h, heart; t, tongue). FIG. 4B: Calsarcin-1 transcripts were detected by radioactive in situ hybridization of a frontal section of an adult mouse heart. Transcripts are detected throughout the atria (a) and ventricles (v). FIG. 4C: Calsarcin transcripts were detected by radioactive in situ hybridization of sections through adult mouse hindlimb muscle. Calsarcin-1 transcripts are localized to soleus (s) and plantaris (p), whereas calsarcin-2 transcripts are localized to the gastrocnemius (g). FIG. 4D: Calsarcin-1 and α-tubulin protein expression was detected by Western blot analysis of extracts from the indicated tissues. FIG. 4E: Calsarcin-1 transcripts were detected by Northern analysis of RNA from C2 cells in growth medium (GM) or differentiation medium (DM) for the indicated days. Scale bar 500 μm.

FIGS. 5A-B—Subcellular localization of calsarcin-1. Neonatal rat cardiomyocytes were analyzed by immunostaining with calsarcin-1 antiserum and antibodies directed against α-actinin (upper panel) and CnA (lower panel). The overlay indicates that calsarcin-1 colocalizes with α-actinin and CnA. Scale bar 10 μm.

FIGS. 6A-C—Coimmunoprecipitation of calsarcins with calcineurin and α-actinin. FIG. 6A: Cos-cells were transiently transfected with expression vectors encoding FLAG-can, FLAG-α-actinin-1, or Myc-calsarcin-1 (Cs-1) and immunoprecipitations were performed. The upper panel shows an anti-FLAG immunoblot of anti-Myc immunoprecipitates and demonstrates the association of CnA and α-actinin with Cs-1. IgG heavy chain also is recognized by the secondary antibody. The middle panel shows an anti-FLAG immunoblot of cell extracts to demonstrate the presence of CnA and α-actinin. The lower panel shows an anti-Myc immunoblot of cell extracts to demonstrate the presence of calsarcins. FIG. 6B: Cos cells were transiently transfected with expression vectors encoding Myc-α-actinin-2, HA-Cs-1 or FLAG-can and immunoprecipitations were performed with anti-Myc antibody followed by immunoblotting with FLAG antibody. The upper panel shows an anti-FLAG immunoblot of anti-Myc immunoprecipitates and demonstrates association of CnA with Cs-1. The second panel from the top shows an anti-FLAG immunoblot of cell extracts to demonstrate the presence of can. The next panel shows an anti-Myc immunoblot to demonstrate the presence of α-actinin and an anti-HA immunblot to demonstrate the presence of Cs-1, respectively. FIG. 6C: Extracts prepared from primary neonatal rat cardiomyocytes were immunoprecipitated with anti-Cs-1 antibody or preimmune serum and analyzed by immunoblotting with anti-α-actinin antibody. α-actinin is specifically immunoprecipitated with anti-Cs-1.

FIG. 7—Mapping of calsarcin-, calcineurin- and α-actinin-interacting domains. N- and C-terminal calsarcin-1 truncations were generated and fused to a Gal4-DNA-binding domain to test their ability to interact with CnA or α-actinin, as assessed by β-gal activity in yeast. Complementary experiments were conducted by coimmunoprecipitation of Myc-tagged calsarcin-1 with FLAG-tagged CnA or α-actinin, respectively. Taken together, amino acids 153-200 appear to be necessary for the interaction with α-actinin, whereas amino acids 217-240 are required for calsarcin's association with CnA.

FIG. 8—A schematic diagram of the sarcomere showing the binding of calsarcin-1 to the Z-disk and its association with calcineurin (CNA).

FIG. 9—Northern blot analysis of calsarcin-3 in adult human and mouse tissues. Calsarcin transcripts were detected by Northern analysis of the indicated human and mouse tissues. Calsarcin-3 mRNA is predominantly detected in skeletal muscle, of both species.

FIG. 10—Coimmunoprecipitation of calsarcins with calcineurin and α-actinin, telethonin and γ-filamin. As demonstrated in FIG. 6 calscarin 1, 2, and 3 interacted with calcineurin and α-actinin, and γ-filamin. Furthermore, by coimmunoprecipation all calsarcins interacted the sacromeric protein of heart and skeletal muscle telethonin. Telethonin is a disease gene involved in limb-girdle muscular dystrophy and may play a role in the stretch-response of striated muscle both in cardiac and skeletal muscle.

FIG. 11—Immunostaining of mouse skeletal muscle with anti-calsarcin-3 antibody confirming z-disc location. Antibody against was raised against calsarcin-3 which shows z-disc staining in skeletal muscle proven by colocalization with α-actinin.

FIG. 12—Overexpression of calsarcin-1 in C2C1 cells promotes (pro) sarcomere formation. Overexpression of calsarcin-1 in C2C12 myoblasts results in early, (after one day of differentiation) and enhanced sarcomere formation

FIG. 13—Alignment of calsarcins 1-3.

### DETAILED DESCRIPTION OF ILLUSTRATIVE EMBODIMENTS

Heart failure—the inability of the heart to pump blood at a rate sufficient to sustain homeostasis—is a major health issue in the world today. This is true not only due to the untimely deaths caused by heart disease, but the tremendous expense incurred due to required patient support, including prolonged hospitalization. Thus, there remains a great need to address this costly and debilitating disease.

The present inventors report herein a calcineurin-associated peptide (calsarcin-1) capable of binding the activated form of calcineurin. In a specific embodiment, calsarcin-1 also binds the inactive form of calcineurin. In addition, calsarcin-1 binds α-actinin (both the sarcomeric and nonsarcomeric forms), which is linked to the sarcomere. The sarcomere is an important muscular subunit in muscle tissues, such as cardiac muscle, which in many ways resembles striated muscle. The sarcomere is the minimum contractile element of muscle and is comprised of protein filaments, including actin filaments and myosin filaments. The thin filaments are protein filaments comprised of smaller actin subunits which combine to form filamentous actin, or F actin. Each thin filament consists of two intertwined actin filaments. The thick filaments are composed of the protein molecule myosin, which has both a tail region and a head region, in which the head regions connect the thick filaments to the thin filaments during contraction. The sarcomere itself is defined as the area between two Z lines, also called Z discs, which are demaractions in which the thin filaments of one sarcomere attaches to the thin filaments of the next sarcomere. As discussed supra, the Z discs are composed of α-actinin.

Current results indicate that the interaction between calsarcin-1 and calcineurin is pertinent to the pathobiology, and ultimately to the therapy, of human heart disease. For example, familial forms of hypertrophic cardiomyopathy are caused by mutations in genes encoding proteins of the sarcomere (Seidman and Seidman, 1998) in a manner that likely involves calcineurin signaling (Marban et al, 1987). Administration of the calcineurin antagonist drugs cyclosporin A or FK-506 prevents cardiac hypertrophy in transgenic animal models of familial forms of hypertrophic cardiomyopathy (Sussman et al, 1998), but the analogous clinical trials are precluded because of toxic side effects (e.g, immunosuppression and hypertension) of existing agents.

Calcineurin antagonists also prevent cardiac hypertrophy and heart failure in some, although not all, animal models of acquired forms of cardiomyopathy that are common in human populations (Sussman et al, 1998; Ding et al, 1999; Zhang et al, 1999), but the same limitations to clinical trials apply. The relative abundance of calsarcin-1 in cardiac muscle makes it a prime target for drug development to circumvent these limitations of current calcineurin antagonists.

Results of the present invention further indicate that calsarcin 1, 2 and 3 are candidate genes for inherited muscular dystrophies and myopathies; and further supports this by the interaction of calsarcains with telethonin, a gene involved in limb-girdle muscular dystrophy. Muscular dystrophy (MD) refers to a group of genetic diseases characterized by progressive weakness and degeneration of the skeletal or voluntary muscles which control movement. The muscles of the heart and some other involuntary muscles are also affected in some forms of MD, and a few forms involve other organs as well. The major forms of MD include myotonic, Duchenne, Becker, limb-girdle, facioscapulohumeral, congenital, oculopharyngeal, distal and Emery-Dreifuss.

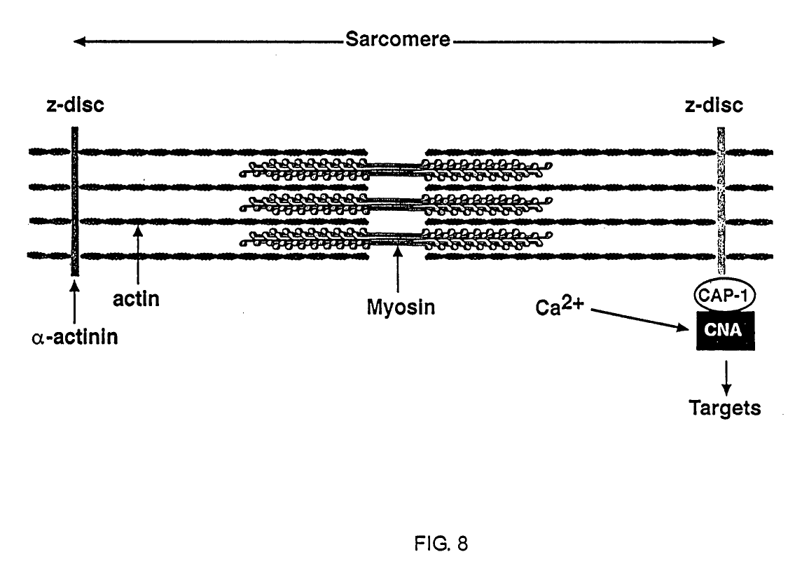

The significance of calcineurin-associated proteins in cardiomyopathies and muscular dystrophies is further indicated in the current invention. Based on their interactions and colocalization in vivo, it also is proposed herein that calsarcin-1 links calcineurin to the Z-band where it can sense changes in calcium signaling in the myocyte and potentially transduce a hypertrophic signal (FIG. 8). Calsarcin-1, and/or other calsarcin proteins, such as calsarcin-2 or calsarcin-3, may also play structural and/or mechanosensory roles in cardiac and skeletal myocytes through modulation of the Z-band and its association with other proteins in the cell. The Z-band has been shown to play important roles in regulating muscle cell structure and function. Thus, calsarcins are likely to be intimately involved in these processes and is a strong candidate for a gene involved in human cardiomyopathies and muscular dystrophies.

I. Calsarcin Peptides and Polypeptides

Applicants provide herein protein sequences for human calsarcin-1 (SEQ ID NO:2) and mouse calsarcin-1 (SEQ ID NO:4), human calsarcin-2 (SEQ ID NO:6), mouse calsarcin-2 (SEQ ID NO:8), human calsarcin-3 (SEQ ID NO:10) and mouse calsarcin-3 (SEQ ID NO:12). In a specific embodiment, a calcineurin associated sarcomeric protein (calsarcin) peptide, a calsarcin polypeptide or a calsarcin protein refer to calsarcin-1, calsarcin-2 or calsarcin-3. In addition to the entire calsarcin-1 molecules, the present invention also relates to fragments of the polypeptides that may or may not retain various of the functions described below. Fragments, including the N-terminus of the molecule, may be generated by genetic engineering of translation stop sites within the coding region (discussed below). Alternatively, treatment of calsarcin-1 with proteolytic enzymes, known as proteases, can produce a variety of N-terminal, C-terminal and internal fragments. Examples of fragments may include contiguous residues of SEQ ID NOS:2, 4, 6, 8, 10, and 12, of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 200 or more amino acids in length. These fragments may be purified according to known methods, such as precipitation (e.g, ammonium sulfate), HPLC, ion exchange chromatography, affinity chromatography (including immunoaffinity chromatography) or various size separations (sedimentation, gel electrophoresis, gel filtration).

A. Structural Features

A skilled artisan is aware of standard methods to determine structural features of calsarcin-1, calsarcin-2 and/or calsarcin-3, such as commercially available computer programs or government-supported programs available on the Internet (http://www.ncbi.nlm.nih.gov/Structure/).

B. Functional Aspects

As described in the Examples herein, a region of calsarcin-1 is involved in binding to α-actinin. In a specific embodiment, this region is localized to between amino acids 105 and 176 (see Example 7). In another embodiment, a region of calsarcin-1 is determined to be involved in binding to calcineurin by similar methods. In an additional embodiment, calsarcin-2 and/or calsarcin-3 are identified to be involved in binding to calcineurin by similar methods. In an alternative embodiment, more than one calsarcin polypeptide interacts with calcineurin, and in a specific embodiment, more than one calsarcin polypeptide interacts with calcineurin concomitantly. In another embodiment, more than one calsarcin polypeptide interacts with α-actinin. In an additional specific embodiment, more than one calsarcin polypeptide interacts with α-actinin concomitantly. In a specific embodiment, calsarcin-1, calsarcin-2, and/or calsarcin-3 amino acid sequences are compared by computer programs standard in the art or with the naked eye to search for similar domains which are likely candidates for calcineurin interaction. This domain in calsarcin-1, calsarcin-2 and/or calsarcin-3 is tested for calcineurin binding by standard methods in the art, such as directed two hybrid analysis or coimmunoprecipitation. Thses studies have revealed that the calsarcin-1 calcineurin binding domain is localized to residues 217-240.

C. Variants of Calsarcin

Amino acid sequence variants of the calsarcin polypeptide can be substitutional, insertional or deletion variants. Deletion variants lack one or more residues of the native protein which are not essential for function or immunogenic activity. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein or polypeptide to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of the biological utility or activity of the corresponding polypeptide, as discussed below. Table 1, provided elsewhere herein, shows the codons that encode particular amino acids.

In making such changes, the hydropathic index of amino acids may be considered. The importance of the hydropathic amino acid index in conferring interactive biological function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of calsarcin, but with altered and even improved characteristics.

D. Domain Switching

As described in the examples, the present inventors isolated calsarcin. Given the homology between human, mouse and rat calsarcin, determined by standard means in the art, an interesting series of mutants can be created by substituting homologous regions of various proteins. This is known, in certain contexts, as “domain switching.”

Domain switching involves the generation of chimeric molecules using different but, in this case, related polypeptides. By comparing various calsarcin proteins, one can make predictions as to the functionally significant regions of these molecules. It is possible, then, to switch related domains of these molecules in an effort to determine the criticality of these regions to calsarcin function. These molecules may have additional value in that these “chimeras” can be distinguished from natural molecules, while possibly providing the same function.

E. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In a specific embodiment a fusion protein comprising calsarcin is utilized to inhibit calcineurin activation of gene transcription in a cell in which the fusion protein localizes said fusion protein calsarcin to a subcellular region other than a subcellular region of normal function for said calcineurin. Methods to identify subcellular regions for localization of calcineurin function are well known in the art and include transmission electron microscopy isolation of labeled calcineurin through subcellular fractionation, and immunolocalization. In a specific embodiment a fusion protein comprising calsarcin also comprises a targeting peptide, wherein the targeting peptide comprises a geranylgeranyl group, a nuclear localization signal, a myristilation signal, or an endoplasmic reticulum signal peptide. In a specific embodiment, a geranylgeranyl group or a myristilation signal target the fusion protein to a membrane.

F. Purification of Proteins

It is desirable to purify calsarcin or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest is further purified using chromatographic and electrophoretic techniques to achieve partial or complete purification (or purification to homogeneity). Analytical methods particularly suited to the preparation of a pure peptide include ion-exchange chromatography, exclusion chromatography; polyacrylamide gel electrophoresis; and isoelectric focusing. Particularly efficient methods of purifying peptides are fast protein liquid chromatography and HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al, 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, and the like.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

G. Synthetic Peptides

The present invention also describes smaller calsarcin peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et al, (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

H. Antigen Compositions

The present invention also provides for the use of calsarcin proteins or peptides as antigens for the immunization of animals relating to the production of antibodies. It is envisioned that calsarcin or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

II. Nucleic Acids

The present invention also provides, in another embodiment, nucleic acids encoding calsarcin. Calsarcin nucleic acids include human calsarcin-1, human calsarcin-2, human calsarcin-3, mouse calsarcin-1, mouse calsarcin-2, and mouse calsarcin-3. Nucleic acids for human calsarcin-1 (SEQ ID NO:1) and mouse calsarcin-1 (SEQ ID NO:3) have been identified. In addition, three mouse calsarcin-2 ESTs and four human calsarcin-2 ESTs were identified (see Example 1). The mouse calsarcin-2 ESTs are as follows: GenBank No. AA036142; GenBank No. AW742494; and GenBank No. W29466. The human calsarcin-2 ESTs are as follows: GenBank No. AW964108; GenBank No. AA197193; GenBank No. AW000988; and GenBank No. AA176945. In a specific embodiment, the mouse calsarcin-2 ESTs and the human calsarcin-2 ESTs are aligned by computer programs known in the art to identify full-length mouse calsarcin-2 and human calsarcin-2 sequences respectively. The present invention is not limited in scope to these nucleic acids. However, one of ordinary skill in the art could, using these nucleic acids, readily identify related homologs in various other species (e.g, rat, rabbit, dog, monkey, gibbon, human, chimp, ape, baboon, cow, pig, horse, sheep, cat and other species).

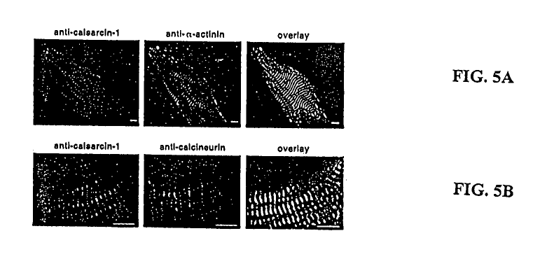

In another specific embodiment, calsarcin-3 was discovered “in silico” by comparing calsarcin 1 and calsarcin 2 sequences with the database. Human genomic DNA (AC 008453.3; public not Celera database) containing several homologous sequences was confirmed to be exons of calsarcin-3. Primers were designed and a human skeletal muscle library was screened for the full-length cDNA for human calsarcin-3 (FIG. 5). Similarly, a mouse skeletal library was screened and several independent and overlapping clones encoding for mouse calscarcin-3 were identified. The full-length nucleic acid sequences from cDNA and genomic libraries are compared to differentiate between exon and intron sequences (Sambrook, et al, 1989). Furthermore, computer programs well known in the art use the nucleic acid sequence to generate a predicted amino acid sequence.

In addition, it should be clear that the present invention is not limited to the specific nucleic acids disclosed herein. As discussed below, a “calsarcin nucleic acid” may contain a variety of different bases and yet still produce a corresponding polypeptide that is functionally indistinguishable, and in some cases structurally, from the human and mouse nucleic acids disclosed herein.

Similarly, any reference to a nucleic acid should be read as encompassing a host cell containing that nucleic acid and, in some cases, capable of expressing the product of that nucleic acid. In addition to therapeutic considerations, cells of cell-free systems expressing nucleic acids of the present invention may prove useful in the context of screening for agents that induce, repress, inhibit, augment, interfere with, block, abrogate, stimulate or enhance the function of calsarcin.

A. Nucleic Acids Encoding Calsarcin-1

Nucleic acids according to the present invention may encode a calsarcin nucleic acid, a domain of calsarcin, or any other fragment of calsarcin-1 as set forth herein. In a preferred embodiment, the nucleic acid encodes a calsarcin peptide, polypeptide or protein which has functional activity or immunogenic activity. In a specific embodiment, the terms “calsarcin nucleic acid” or “calsarcin” refer to a calsarcin-1, calsarcin-2 or calsarcin-3 nucleic acid, a domain of calsarcin-1, calsarcin-2 or calsarcin-3, respectively, or any other fragment of calsarcin-1, calsarcin-2 or calsarcin-3 as set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given calsarcin from a given species may be represented by natural variants that have slightly different nucleic acid sequences but, nonetheless, encode the same protein (see Table 1 below).

As used in this application, the term “a nucleic acid encoding calsarcin” refers to a calsarcin nucleic acid molecule that has been isolated free of total cellular nucleic acid. In preferred embodiments, the invention concerns a nucleic acid sequence essentially as set forth in SEQ ID NOS:1, 3, 5, 7, 9, or 11. The term “as set forth in SEQ ID NOS:1, 3, 5, 7, 9, or 11” means that the nucleic acid sequence substantially corresponds to a portion of SEQ ID NO:1, 3, 5, 7, 9, or 11 respectively. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NOS: 1, 3, 5, 7, 9, or 11 are contemplated. Sequences that are essentially the same as those set forth in SEQ ID NOS:1, 3, 5, 7, 9, or 11 also may be functionally defined as sequences that are capable of hybridizing to a nucleic acid segment containing the complement of SEQ ID NOS:1, 3, 5, 7, 9, or 11 respectively, under standard conditions.

The DNA segments of the present invention include those encoding biologically functional equivalent calsarcin proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the sequence set forth in SEQ ID NOS:1, 3, 5, 7, 9, or 11. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NOS:1, 3, 5, 7, 9, or 11, respectively, under relatively stringent conditions such as those described herein. Such sequences may encode the entire calsarcin polypeptides or proteins, or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, or 3000 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C, while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

One method of using probes and primers of the present invention is in the search for genes related to calsarcin or, more particularly, homologs of calsarcin from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to exon/intron splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g, ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al, 1987; Forster and Symons, 1987). For example, a large number of ribozymes accelerate phosphoester transfer reactions with a high degree of specificity, often cleaving only one of several phosphoesters in an oligonucleotide substrate (Cook et al, 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al, 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al, 1991; Sarver et al, 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. Vectors for Cloning, Gene Transfer and Expression

Within certain embodiments expression vectors are employed to express a calsarcin polypeptide product, which can then be purified and, for example, be used to vaccinate animals to generate antisera or monoclonal antibody with which further studies may be conducted. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the native calsarcin promoter will be employed to drive expression of either the corresponding calsarcin nucleic acid, a heterologous calsarcin nucleic acid, a screenable or selectable marker nucleic acid, or any other nucleic acid of interest.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof. Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et al.; |
| | 1990 |
| Immunoglobulin Light Chain | Queen et al., 1983; Picard et al., 1984 |
| T-Cell Receptor | Luria et al., 1987; Winoto et al., 1989; Redondo et al.; 1990 |
| HLA DQ a and/or DQ | Sullivan et al., 1987 |
| -Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| -Actin | Kawamoto et al., 1988; Ng et al.; 1989 |
| Muscle Creatine Kinase (MCK) | Jaynes et al., 1988; Horlick et al., 1989; Johnson et al., 1989 |
| Prealbumin (Transthyretin) | Costa et al., 1988 |
| Elastase I | Ornitz et al., 1987 |
| Metallothionein (MTII) | Kerin et al., 1987; Culotta et al., 1989 |
| Collagenase | Pinkert et al., 1987; Angel et al., 1987a |
| Albumin | Pinkert et al., 1987; Tronche et al., 1989, 1990 |
| -Fetoprotein | Godbout et al., 1988; Campere et al., 1989 |
| t-Globin | Bodine et al., 1987; Perez-Stable et al., 1990 |
| Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| ₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., 1985; |
| | Firak et al., 1986; Herr et al., 1986; Imbra et al., 1986; |
| | Kadesch et al., 1986; Wang et al., 1986; Ondek et al., 1987; |
| | Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., 1983; |
| | de Villiers et al., 1984; Hen et al., 1986; Satake et al., 1988; |
| | Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek et |
| | al., 1986; Celander et al., 1987; Thiesen et al., 1988; |
| | Celander et al., 1988; Choi et al., 1988; Reisman et al., 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., 1987; |
| | Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et al., |
| | 1988; Feng et al., 1988; Takebe et al., 1988; Rosen et al., |
| | 1988; Berkhout et al., 1989; Laspia et al., 1989; Sharp et al., |
| | 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; Haslinger et |
| | Heavy metals | al., 1985; Searle et al., 1985; Stuart |
| | | et al., 1985; Imagawa et al., 1987, |
| | | Karin et al., 1987; Angel et al., |
| | | 1987b; McNeall et al., 1989 |
| MMTV (mouse mammary | Glucocorticoids | Huang et al., 1981; Lee et al., 1981; |
| tumor virus) | | Majors et al., 1983; Chandler et al., |
| | | 1983; Lee et al., 1984; Ponta et al., |
| | | 1985; Sakai et al., 1988 |
| -Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle Disease | Hug et al., 1988 |
| | Virus | |
| GRP78 Gene | A23187 | Resendez et al., 1988 |
| -2-Macroglobulin | IL-6 | Kunz et al., 1989 |
| Vimentin | Serum | Rittling et al., 1989 |
| MHC Class I Gene H-2b | Interferon | Blanar et al., 1989 |
| HSP70 | ElA, SV40 Large T Antigen | Taylor et al., 1989, 1990a, 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone Gene | | |

Of particular interest are muscle specific promoters, and more particularly, cardiac specific promoters. These include the myosin light chain-2 promoter (Franz et al, 1994; Kelly et al, 1995), the α actin promoter (Moss et al, 1996), the troponin 1 promoter (Bhavsar et al, 1996); the Na⁺/Ca²⁺ exchanger promoter (Bames et al, 1997), the dystrophin promoter (Kimura et al, 1997), the creatine kinase promoter (Ritchie, 1996), the α7 integrin promoter (Ziober and Kramer, 1996), the brain natriuretic peptide promoter (LaPointe et al, 1996) and the α B-crystallin/small heat shock protein promoter (Gopal-Srivastava et al, 1995).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

(iv) Bidirectional Promoters

In other embodiments of the present invention, a bidirectional promoter is utilized to create multiple species of messages. For example, the aldehyde reductase bidirectional promoter (Barski et al, 1999) is capable of generating transcription in opposite directions to stoichiometric levels. Thus, a skilled artisan may utilize a promoter such as the bidirectional aldehyde reductase promoter to simultaneously generate two species of messages while concomitantly conserving on space required to be present or cloned into an expression vector. The gene product generated by the bidirectional promoter could be RNA or protein, and the bidirectional promoter could transcribe a reporter gene message and a calsarcin message, calcineurin message, or α-actinin message, in addition to any sequence of interest.

(v) Reporter Sequences

The term “reporter sequence” as used herein is defined as the nucleotide sequence which when expressed can be detected. The expressed product itself can be detected, such as an RNA or protein, or a metabolite or other characteristic secondarily affected by the reporter product can be detected. The skilled artisan recognizes that any reporter gene that could be detected by transcutaneous monitoring, by visualization with UV light, by visualization with infrared light, or by visualization with other imaging techniques, such as X-ray or MRI, would be of obvious value. Any tissue or body fluid or cell culture or cell free extract is sampled depending on the marker used. For example, fluorescence, colorimetric assays, secreted proteins, histological markers, visible changes in a transgenic animal and other markers used by those skilled in the art may be utilized to reflect the expression of a specific nucleic acid. Examples of reporter sequences include chloramphenicol acetyltransferase (CAT), green fluorescent protein (GFP), enhanced GFP, blue fluorescent protein, β-galactosidase, β-glucuronidase and luciferase. In a specific embodiment a reporter gene containing an epitope tag is monitored.

(vi) Delivery of Expression Vectors

One of the therapeutic embodiments contemplated by the present inventors is the intervention, at the molecular level, in the events involved in cardiac failure. Specifically, the present inventors intend to provide, to a cardiac cell, an expression construct capable of providing a calsarcin to that cell. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below. Various routes are contemplated, but local provision to the heart and systemic provision (intraarterial or intravenous) are preferred.

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grinhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al, 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevac, 1991). In nature, adenovirus can package approximately 105% of the wild-type genome (Ghosh-Choudhury et al, 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g, Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g, 109-1012 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al, 1963; Top et al, 1971), demonstrating their safety and therapeutic potential as in vivo gene transfer vectors.

Adenovirus vectors have been used in eukaryotic gene expression (Levrero et al, 1991; Gomez-Foix et al, 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al, 1990; Rich et al, 1993). Studies in administering recombinant adenovirus to different tissues include trachea instillation (Rosenfeld et al, 1991; Rosenfeld et al, 1992), muscle injection (Ragot et al, 1993), peripheral intravenous injections (Herz and Gerard, 1993) and stereotactic inoculation into the brain (Le Gal La Salle et al, 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al, 1983). When a recombinant plasmid containing a cDNA, together with the retroviral LTR and packaging sequences is introduced into this cell line (by calcium phosphate precipitation, for example), the packaging sequence allows the RNA transcript of the recombinant plasmid to be packaged into viral particles, which are then secreted into the culture media (Nicolas and Rubenstein, 1988; Temin, 1986; Mann et al, 1983). The media containing the recombinant retroviruses is then collected, optionally concentrated, and used for gene transfer. Retroviral vectors are able to infect a broad variety of cell types. However, integration and stable expression require the division of host cells (Paskind et al, 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al, 1989). Using antibodies against major histocompatibility complex class I and class II antigens, they demonstrated the infection of a variety of human cells that bore those surface antigens with an ecotropic virus in vitro (Roux et al, 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al, 1981). Another concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al, 1988; Hersdorffer et al, 1990).

Other viral vectors may be employed as expression constructs in the present invention. Vectors derived from viruses such as vaccinia virus (Ridgeway, 1988; Baichwal and Sugden, 1986; Coupar et al, 1988). adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al, 1988; Horwich et al, 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al, 1990). This suggested that large portions of the genome could be replaced with foreign genetic material. The hepatotropism and persistence (integration) were particularly attractive properties for liver-directed gene transfer. Chang et al, recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al, 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al, 1990) DEAE-dextran (Gopal, 1985), electroporation (Tur-Kaspa et al, 1986; Potter et al, 1984), direct microinjection (Harland and Weintraub, 1985), DNA-loaded liposomes (Nicolau and Sene, 1982; Fraley et al, 1979) and lipofectamine-DNA complexes, cell sonication (Fechheimer et al, 1987), gene bombardment using high velocity microprojectiles (Yang et al, 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al, 1987). Several devices for accelerating small particles have been developed. One such device relies on a high voltage discharge to generate an electrical current, which in turn provides the motive force (Yang et al, 1990). The microprojectiles used have consisted of biologically inert substances such as tungsten or gold beads.

Selected organs including the liver, skin, and muscle tissue of rats and mice have been bombarded in vivo (Yang et al, 1990; Zelenin et al, 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al, 1989). In other embodiments, the liposome may be complexed or employed in conjunction with nuclear non-histone chromosomal proteins (HMG-1) (Kato et al, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al, 1990). Recently, a synthetic neoglycoprotein, which recognizes the same receptor as ASOR, has been used as a gene delivery vehicle (Ferkol et al, 1993; Perales et al, 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

F. Nucleic Acid Detection

Nucleic acid used is isolated from cells contained in the biological sample, according to standard methodologies (Sambrook et al, 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g, ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, 3H, or other label), with a fluorophore (rhodamine, fluorescein) or a chemilumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al, 1990, each of which is incorporated herein by reference in its entirety.

Briefly, in PCR, two primer sequences are prepared that are complementary to regions on opposite complementary strands of the marker sequence. An excess of deoxynucleoside triphosphates are added to a reaction mixture along with a DNA polymerase, e.g, Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPA No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al, (1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al, 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the calsarcin genes that may then be analyzed by direct sequencing.

(vi) Kit Components

All the essential materials and reagents required for detecting and sequencing a calsarcin and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

III. Generating Antibodies Reactive with Calsarcin

In another aspect, the present invention contemplates an antibody that is immunoreactive with a calsarcin molecule of the present invention, or any portion thereof. An antibody can be a polyclonal or a monoclonal antibody. In a preferred embodiment, an antibody is a monoclonal antibody. Means for preparing and characterizing antibodies are well known in the art (see, e.g, Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to calsarcin-related antigen epitopes. Additionally, it is proposed that monoclonal antibodies specific to the particular calsarcin of different species may be utilized in other useful applications

In general, both polyclonal and monoclonal antibodies against calsarcin may be used in a variety of embodiments. For example, they may be employed in antibody cloning protocols to obtain cDNAs or genes encoding other calsarcins. They may also be used in inhibition studies to analyze the effects of calsarcin-related peptides in cells or animals. Calsarcin antibodies will also be useful in immunolocalization studies to analyze the distribution of calsarcin during various cellular events, for example, to determine the cellular or tissue-specific distribution of calsarcin polypeptides, respectively, under different points in the cell cycle. A particularly useful application of such antibodies is in purifying native or recombinant calsarcin, for example, using an antibody affinity column. The operation of all such immunological techniques will be known to those of skill in the art in light of the present disclosure.

Means for preparing and characterizing antibodies are well known in the art (see, e.g, Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g, a purified or partially purified calsarcin protein, polypeptide or peptide or cell expressing high levels of calsarcin. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Antibodies of the present invention can be used in characterizing the calsarcin content of healthy and diseased tissues, through techniques such as ELISAs and Western blotting. This may provide a screen for the presence or absence of cardiomyopathy or as a predictor of heart disease.

The use of antibodies of the present invention in an ELISA assay is contemplated. For example, anti-calsarcin-1 or anti-calsarcin-2 antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for calsarcin-1 that differs from the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g, incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g, using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

IV. Combined Therapy

In many clinical situations, it is advisable to use a combination of distinct therapies. Thus, it is envisioned that, in addition to the therapies described herein, one would also wish to provide to the patient more “standard” pharmaceutical cardiac therapies. Examples of standard therapies include so-called “beta blockers”, anti-hypertensives, cardiotonics, anti-thrombotics, vasodilators, hormone antagonists, endothelin antagonists, calcium channel blockers, phosphodiesterase inhibitors, angiotensin type 2 antagonists and cytokine blockers/inhibitors. Also envisioned are combinations with pharmaceuticals identified according to the screening methods described herein.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of calsarcin, or the other agent will be desired. Various combinations may be employed, where calsarcin is “A” and the other agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated as well.

V. Formulations and Routes for Administration to Patients

Where clinical applications are contemplated, it will be necessary to prepare pharmaceutical compositions—expression vectors, virus stocks and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

VI. Methods of Making Transgenic Mice

A particular embodiment of the present invention provides transgenic animals that contain calsarcin-related constructs. Transgenic animals expressing calsarcin, recombinant cell lines derived from such animals, and transgenic embryos may be useful in methods for screening for and identifying agents that interact with calsarcin, respectively, modulate binding of calsarcin to α-actinin, telethonin, or calcineurin or affect cardiac hypertrophy or heart failure through utilization of calsarcin. The use of constitutively expressed calsarcin provides a model for over- or unregulated expression, compared to normal basal expression levels. Also, transgenic animals which are “knocked out” for calsarcin are utilized, such as for screening methods or as models for therapeutic assays for candidate compounds.

A. Methods of Producing Transgenics

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds, Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); in The Qiagenologist, Application Protocols, 3rd edition, published by Qiagen, Inc, Chatsworth, Calif.; and in Sambrook et al. Molecular Cloning: A Laboratory Manual (Cold Spring Harbor Laboratory, Cold Spring Harbor, N.Y., 1989), all of which are incorporated by reference herein.

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

VII. Screening Assays

Thus, the present invention also contemplates the screening of compounds for various abilities to interact with and/or affect calcineurin, telethonin, or α-actinin binding with calsarcin. Particularly preferred compounds will be those useful in inhibiting or promoting the binding of calsarcin to calcineurin. In the screening assays of the present invention, the candidate substance may first be screened for basic biochemical activity—e.g, binding to a target molecule—and then tested for its ability to inhibit modulate expression, at the cellular, tissue or whole animal level.

A. Modulators and Assay Formats

The term “modulating” as used herein is defined as affecting, regulating, influencing, moderating or controlling in any manner an activity of a calcineurin polypeptide. In a preferred embodiment, calcineurin function to act as a serine/threonine protein phosphatase is modulated by administration of calsarcin.

As used herein, the term “candidate substance” refers to any molecule that may potentially modulate calsarcin activity or calsarcin binding to calcineurin, telethonin, or α-actinin. The candidate substance may be a protein or fragment thereof, a small molecule inhibitor, or even a nucleic acid molecule. It may prove to be the case that the most useful pharmacological compounds will be compounds that are structurally related to compounds which interact naturally with calsarcin. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like calsarcin, and then design a molecule for its ability to interact with that of calcineurin, α-actinin, or telethonin. Alternatively, one could design a partially functional fragment of calsarcin (binding but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

In this case, there is ample evidence that demonstrates the binding of calsarcin to calcineurin, telethonin, or α-actinin. By analyzing the binding of calsarcin to this target molecule, much information can be gleaned about the ability of calsarcin to recognize calcineurin, telethonin, or α-actinin. With this information, predictions can be made regarding the structure of potential inhibitors of calcineurin activity or activators or facilitators of calsarcin binding to calcineurin or α-actinin.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g, peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of hypertrophic response.

Other suitable inhibitors include antisense molecules, ribozymes, and antibodies (including single chain antibodies), each of which would be specific for a target located within the calcineurin pathway. Such compounds are described in greater detail elsewhere in this document.

It will, of course, be understood that all the screening methods of the present invention are useful in themselves notwithstanding the fact that effective candidates may not be found. The invention provides methods for screening for such candidates, not solely methods of finding them.

In accordance with an object of the present invention there is a method to screen for a modulator of calsarcin binding to calcineurin comprising providing a calsarcin, respectively, and calcineurin, admixing them in the presence of a candidate modulator, measuring calsarcin/calcineurin binding, and comparing the binding with the binding of calsarcin, respectively, and calcineurin in the absence of the candidate modulator. The difference in binding of calsarcin and calcineurin in the presence versus absence of the candidate modulator identifies the candidate modulator as a modulator of calsarcin, respectively, binding to calcineurin. A skilled artisan is aware this could be performed in a cell free system or within an intact cell. In specific embodiments the intact cell is a myocyte, H9C2 cell, C2C12 cell, a 3T3 cell, a 293 cell, a neonatal cardiomyocyte cell or a myotube cell. Preferably the cell is in an animal. Although the modulator can increase or decrease calsarcin binding to calcineurin, it is preferred that the candidate modulator increases binding of calsarcin to calcineurin.

In other specific embodiments of the present invention, the binding is measured by easily detectable means. This includes fluorescence, radioactivity, by detecting close physical proximity, immunological detection, colorimetric assay or transactivation of a reporter gene.

Where applicable, both of the calsarcin and/or calcineurin are labeled, such as with a quenchable label and a quenching agent, as in fluorescence assays. Such a method to assay for binding in the presence or absence of a candidate modulator may in specific embodiments utilize the premise of a two hybrid assay.

B. In Vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays.

In one embodiment of this kind, the screening of compounds that bind to a calcineurin or calsarcin molecule or fragment thereof is provided.

The target may be either free in solution, fixed to a support, expressed in or on the surface of a cell. Examples of supports include nitrocellulose, a column or a gel. Either the target or the compound may be labeled, thereby permitting determining of binding. In another embodiment, the assay may measure the inhibition of binding of a target to a natural or artificial substrate or binding partner (such as calsarcin). Competitive binding assays can be performed in which one of the agents (calsarcin, for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, a calsarcin and washed. Bound polypeptide is detected by various methods.

Purified target, such as calcineurin, α-actinin, telethonin, calsarcin-1, calsarcin-2 or calsarcin-3, can be coated directly onto plates or supports for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region (e.g, amino acids 105 to 176) to a solid phase, or support.

Thus, there is provided herein a method to identify a peptide which binds calsarcin by attaching a calsarcin polypeptide, respectively, or a fragment thereof, to a support, exposing the polypeptide or fragment to a candidate peptide, and assaying for binding of the candidate peptide to the polypeptide or fragment. The binding is assayed by any standard means in the art, such as through radioactivity, immunologic detection, fluorescence, gel electrophoresis or colorimetry means. In a specific embodiment, additional calsarcins are identified wherein calcineurin is attached to a support and subject to analagous assays.

C. In Cyto Assays

Various cell lines that express calsarcin can be utilized for screening of candidate substances. For example, cells containing calsarcin with an engineered indicator can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (growth, size, Ca-⁺⁺ effects). Alternatively, molecular analysis may be performed in which the function of calsarcin and related pathways may be explored. This involves assays such as those for protein expression, enzyme function, substrate utilization, mRNA expression (including differential display of whole cell or polyA RNA) and others.

Thus, in accordance with the present invention there is provided herein a method of screening for a candidate substance for anti-cardiomyopic hypertrophy activity or anti-heart failure activity by providing a cell lacking a functional calsarcin polypeptide, contacting the cell with a candidate substance and determining the effect of the candidate substance on the cell. The cell lacking a functional calsarcin polypeptide is described elsewhere herein and may derive from a transgenic non-human animal containing the cell, as in a cell line. The cell is preferably a muscle cell and may have a mutation in a regulatory region of calsarcin, such as a deletion, insertion or point mutation, or in the coding region, such as a deletion, insertion, frameshift, nonsense, missense or splicing mutation. The cell may be contacted in vitro or in vivo by methods well known in the art, and in a specific embodiment is located in a non-human transgenic animal.

D. In Vivo Assays

The present invention particularly contemplates the use of various animal models. Transgenic animals may be generated with constructs that permit calsarcin expression and activity to be controlled and monitored. The generation of these animals has been described elsewhere herein.

Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

E. Two Hybrid Screens

The term “two hybrid screen” as used herein refers to a screen to elucidate or characterize the function of a protein by identifying other proteins with which it interacts. The protein of unknown function, herein referred to as the “bait” is produced as a chimeric protein additionally containing the DNA binding domain of GAL4. Plasmids containing nucleotide sequences which express this chimeric protein are transformed into yeast cells, which also contain a representative plasmid from a library containing the GAL4 activation domain fused to different nucleotide sequences encoding different potential target proteins. If the bait protein physically interacts with a target protein, the GAL4 activation domain and GAL4 DNA binding domain are tethered and are thereby able to act conjunctively to promote transcription of a reporter gene. If no interaction occurs between the bait protein and the potential target protein in a particular cell, the GAL4 components remain separate and unable to promote reporter gene transcription on their own. One skilled in the art is aware that different reporter genes can be utilized, including β-galactosidase, HIS3, ADE2, or URA3. Furthermore, multiple reporter sequences, each under the control of a different inducible promoter, can be utilized within the same cell to indicate interaction of the GAL4 components (and thus a specific bait and target protein). A skilled artisan is aware that use of multiple reporter sequences decreases the chances of obtaining false positive candidates. Also, alternative DNA-binding domain/activation domain components may be used, such as LexA. One skilled in the art is aware that any activation domain may be paired with any DNA binding domain so long as they are able to generate transactivation of a reporter gene. Furthermore, a skilled artisan is aware that either of the two components may be of prokaryotic origin, as long as the other component is present and they jointly allow transactivation of the reporter gene, as with the LexA system.

Two hybrid experimental reagents and design are well known to those skilled in the art (see “The Yeast Two-Hybrid System” by P. L. Bartel and S. Fields (eds.) (Oxford University Press, 1997), including the most updated improvements of the system (Fashena et al, 2000). A skilled artisan is aware of commercially available vectors, such as the Matchmaker™ Systems from Clontech (Palo Alto, Calif.) or the HybriZAP® 2.1 Two Hybrid System (Stratagene; La Jolla, Calif.), or vectors available through the research community (Yang et al, 1995; James et al, 1996). In alternative embodiments, organisms other than yeast are used for two hybrid analysis, such as mammals (Mammalian Two Hybrid Assay Kit from Stratagene (La Jolla, Calif.)) or E. coli (Hu et al, 2000).

In an alternative embodiment, a two hybrid system is utilized wherein protein-protein interactions are detected in a cytoplasmic-based assay. In this embodiment, proteins are expressed in the cytoplasm, which allows posttranslational modifications to occur and permits transcriptional activators and inhibitors to be used as bait in the screen. An example of such a system is the CytoTrap® Two-Hybrid System from Stratagene™ (La Jolla, Calif.), in which a target protein becomes anchored to a cell membrane of a yeast which contains a temperature sensitive mutation in the cdc25 gene, the yeast homolog for hSos (a guanyl nucleotide exchange factor). Upon binding of a bait protein to the target, hSos is localized to the membrane, which allos activation of RAS by promoting GDP/GTP exchange. RAS then activates a signaling cascade which allows growth at 37° C. of a mutant yeast cdc25H. Vectors (such as pMyr and pSos) and other experimental details are available for this system to a skilled artisan through Stratagene (La Jolla, Calif.). (See also, for example, U.S. Pat. No. 5,776,689, herein incorporated by reference).

Thus, in accordance with an embodiment of the present invention, there is a method of screening for a peptide which interacts with calsarcin comprising introducing into a cell a first nucleic acid comprising a DNA segment encoding a test peptide, wherein the test peptide is fused to a DNA binding domain, and a second nucleic acid comprising a DNA segment encoding at least part of calsarcin, respectively, wherein the at least part of calsarcin, respectively, is fused to a DNA activation domain. Subsequently, there is an assay for interaction between the test peptide and the calsarcin polypeptide or fragment thereof by assaying for interaction between the DNA binding domain and the DNA activation domain. In a preferred embodiment, the assay for interaction between the DNA binding and activation domains is activation of expression of □-galactosidase.

VII. Methods to Treat Cardiac-Related Medical Conditions

The calsarcin-1, calsarcin-2 or calsarcin-3 polypeptide provided herein binds calcineurin, and α-actinin and is associated with hypertrophic cardiomyopathy. The ability to bind calcineurin provides an opportunity to target therapy utilizing calsarcin-1, calsarcin-2 or calsarcin-3, particularly to exploit its high level of expression in cardiac muscle, expression at lower levels in skeletal muscle, and lack of detectability in other tissues. The inhibition of calcineurin activity via presently used therapies such as cyclosporine and FK506 has undesirable side effects due to immunosuppression. Thus, a skilled artisan is provided herein methods to modulate calcineurin activity or to treat cardiac hypertrophy, heart failure or Type II diabetes by administering to an organism suffering therefrom a calsarcin polypeptide or nucleic acid encoding a calsarcin polypeptide. Therefore, it is intended to perturb calcineurin activity by intervening with its function or activity by binding it to, preferably, calsarcin polypeptide present in levels over normal, basal levels. This could be achieved by administering wild-type or mutant forms, such as a dominant negative form, of calsarcin as a means to mislocalize and potentially inhibit calcineurin activity. The term “dominant negative” as used herein refers to a form of calsarcin which disturbs the function of a wild-type form in the same cell. Thus, a dominant negative form of calsarcin may bind to calcineurin and promote aberrant activity of calcineurin, such as through subcellular mislocalization. In a preferred embodiment the calsarcin administered binds up, or titrates away, calcineurin in the cell, thereby reducing the consequent effects of calcineurin, such as facilitating cardiomyopic hypertrophy.

In a specific embodiment, the nucleic acid encoding the calsarcin polypeptide or calcineurin binding fragment thereof is expressed specifically in muscle cells, such as with a muscle-specific promoter. In a specific embodiment, a dominant negative form of calsarcin is administered.

In other methods, there is inhibition of calcineurin activation of gene transcription in a cell by providing to the cell a fusion protein comprising calsarcin or a calcineurin binding fragment thereof, fused to a targeting peptide that localizes the fusion protein to a subcellular region other than where it exerts its function. That calcineurin can sense changes in contractility strongly suggests that its localization to the sarcomere enablies it to respond to calcium alterations due to contraction. Fusion proteins are discussed elsewhere herein. The gene transcription which is affected by such methods may be inhibited by direct means or indirectly, as with inhibiting an upstream effector. In specific embodiments, the gene transcription by calcineurin which is inhibited includes but is not limited to genes encoding cytokines such as IL-2, fetal cardiac genes such as atrial natriuretic factor (ANF), b-type natriuretic peptide (BNP), α-major histocompatibility complex (MHC), and α-skeletal actin. Basic models of NFAT activation discussed supra show transduction of Ca²⁺ signals via calcineurin in many cell types and control of transcription of diverse sets of target genes unique to each cellular environment (Timmerman et al, 1996).

In specific embodiments, therapy with traditional drugs or compounds is utilized in addition to the methods described herein, including administering to an animal a compound selected from the group consisting of an ionotrope, a beta blocker, an antiarrhythmic, a diuretic, a vasodilator, a hormone antagonist, an endothelin antagonist, an angiotensin type 2 antagonist and a cytokine inhibitor/blocker.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Yeast Two-Hybrid Screens. A full-legnth mouse CnA-α cDNA, fused to the GAL4 DNA binding domain was used as bait in a two-hybrid screen of approximately 1.5×10⁶ clones of a human heart cDNA library (Clontech), as described previously (Molkentin et al, 1998). From this screen, the inventors identified a cDNA encoding calsarcin-1. Additional two-hybrid screens of the same cDNA library were performed using calsarcin-1 and calsarcin-2 as bait.

Northern blot analysis. Northern blots of RNA from human and mouse multiple tissues (Clontech) as well as from C2C12 cell extracts were performed as described (Spencer et al, 2000).

Generation of calsarcin antiserum and western blots. A rabbit antiserum was generated against the complete open reading frame of calsarcin-1 fused in-frame with GST. IgG was purified from rabbit serum and used for Western blotting and immunostaining.

Radioactive In Situ Hybridization. RNA probes corresponding to the sense and antisense strains of calsarcin-1 and calsarcin-2 cDNAs were prepared using T7 and T3 RNA polymerase (Roche) and ³⁵S-labeled UTP. Sections of mouse embryos and adult hind limbs were subjected to in situ hybridization, as described previously (Lu et al, 1998).

Cell culture, transfections and immunoprecipitations. Cos-7 cells were maintained in DMEM containing 10% FBS. 2×10⁵ cells were transfected with 1 μg of expression plasmids for full-length and truncated forms of calsarcin-1 and calsarcin-2, CnA and α-actinin-2 using FuGENE 6 reagent (Roche). Calsarcin peptides were fused with an N-terminal HA-epitope or a C-terminal Myc-epitope, α-actinin-2 was fused with N-terminal Myc- or FLAG-epitopes and CnA constructs were fused with an N-terminal FLAG epitope. Forty-eight hours after transfection, cells were harvested in ELB-buffer, containing 50 mM Hepes (pH 7.0), 250 mM NaCl, 5 mM EDTA, 1 mM DTT, 1 mM PMSF and protease inhibitors (Complete; Roche). Cells were briefly sonicated and debris was removed by centrifugation. Tagged proteins were immunoprecipitated for 2-3 hours at 4° C. using protein A/B agarose and 1 μg of the appropriate antibody (monoclonal anti-FLAG, monoclonal anti-Myc and polyclonal anti-Myc). Subsequently, the pellet was washed with ELB-buffer and subjected to SDS-PAGE, transferred to polyvinylidene membranes and immunoblotted using anti-FLAG, anti-Myc or anti-HA-antibodies, respectively.

Immunostaining. The subcellular localization of calsarcin-1, α-actinin and can was determined in neonatal rat cardiomyocytes which were harvested and maintained as described (Molkentin et al, 1998). Immunostaining was performed as described (Spencer et al, 2000). The following antibodies were used: anti-calsarcin-1, anti-sarcomeric α-actinin (Sigman), anti-can (Sigma, Transduction Laboratories, Santa Cruz); secondary antibodies; Anti-mouse/rabbit, Texas red and FITC-labeled (Vectorlabs), respectively. Cryosections of mouse heart and skeletal muscle were fixed in 3.7% formalin for 3 minutes, permeabilized in 0.3% Triton X-100 for 5 minutes and subsequently stained as described above.

Mapping of calsarcin-1 interaction domains. Several N- and C-terminal truncations of calsarcin-1 were fused in-frame with the GAL4 DNA-binding domain in vector pAS1. CnA and α-actinin were fused with the GAL4 transactivation domain in the two-hybrid vector pACT2. Since both full-length and constitutively active CnA displayed background β-galactosidase activity when transfected alone, a mutated CnA, lacking enzymatic activity (Shibasaki et al, 1996) was used in subsequent experiments and did not display any background signal. Calsarcin-1 constructs were transformed with can, α-actinin or pACT2 (as negative control) and grown on appropriate selective medium for 3 days. β-galactosidase activity was determined with filter-lift assays as described (Fields & Song, 1989) and monitored for 1-4 h. Since several C-terminal truncations of calsarcin-1 exhibited β-galactosidase activity when cotransformed with pACT2, complementary coimmunoprecipitation experiments were performed to further define calsarcin's interaction domains for CnA and α-actinin, as described above.

### Example 2

### Identification of Calcineurin-Associated Proteins

To identify proteins which associated with calcineurin, and preferably which were cardiac-specific, two hybrid analysis was performed in yeast for proteins encoded by mouse heart cDNA libraries. In a specific embodiment, the catalytic region of calcineurin is fused to the DNA binding domain of yeast GAL4. From these screens, a muscle-specific calcineurin-associated protein (calsarcin)-1 was identified that associates with calcineurin. Subsequent experiments in mammalian cells demonstrated that calsarcin-1 and calcineurin can form a complex in vivo (see Examples below).

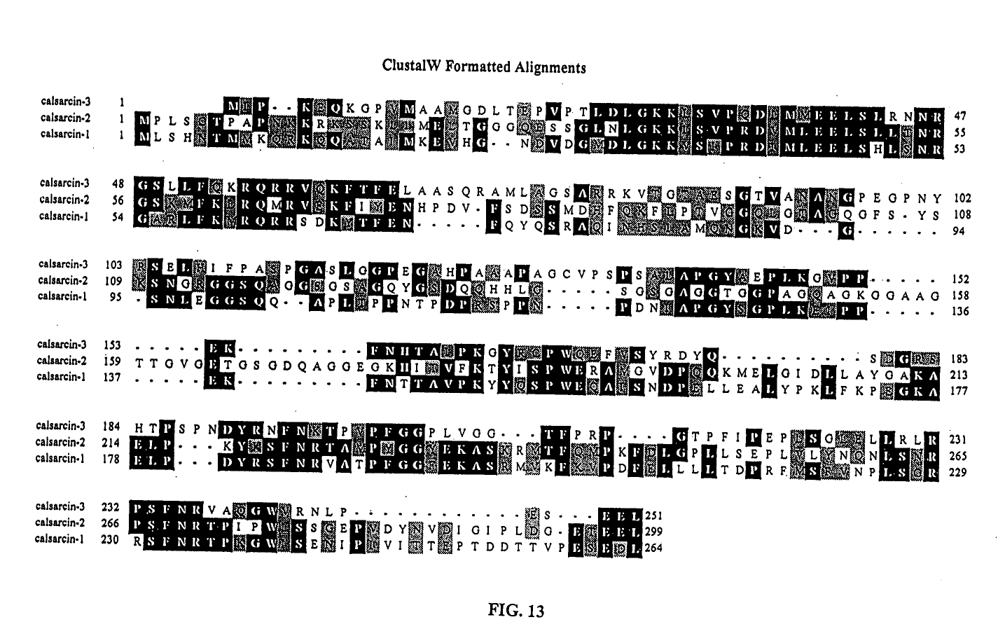

Searching public expressed sequence tag (EST) databases with the mouse calsarcin-1 cDNA sequence, human calsarcin-1 cDNA clones were identified, as well as human and mouse sequences for the related genes calsarcin-2 and calsarcin-3. A skilled artisan is aware of databases available for such searching of both protein and nucleic acid sequences, including GenBank (http://www.ncbi.nlm.nih.gov/Genbank/GenbankSearch.html) or commercially available databases (Celera Genomics, Inc.; Rockville, Md.; www.celera.com). Alignment of calsarcins 1-3 is demonstrated in FIG. 13.

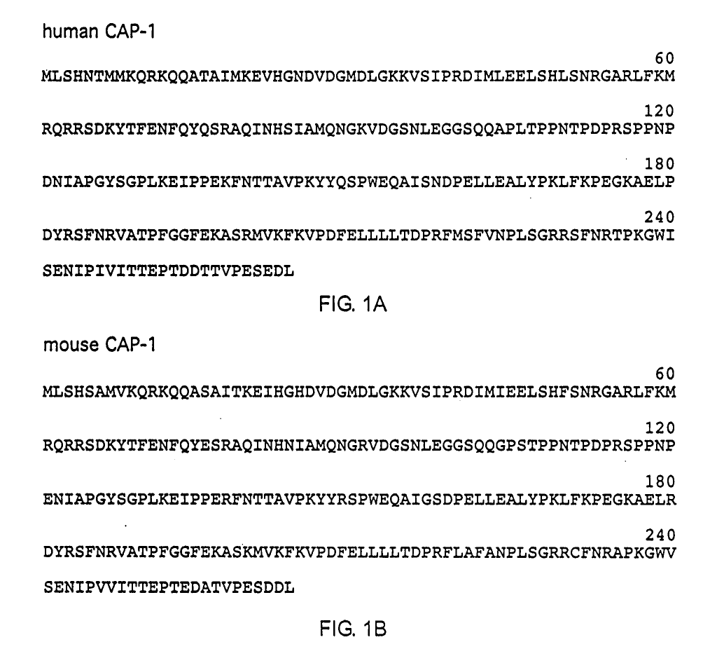

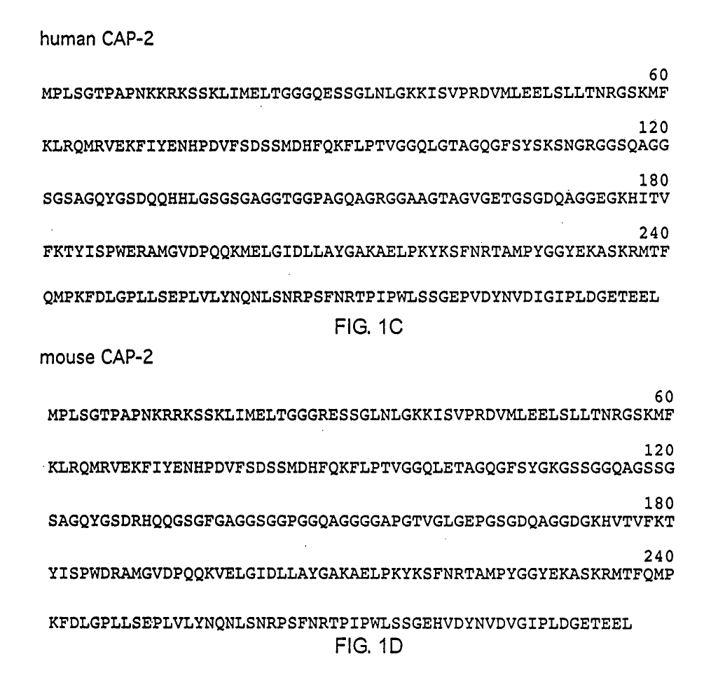

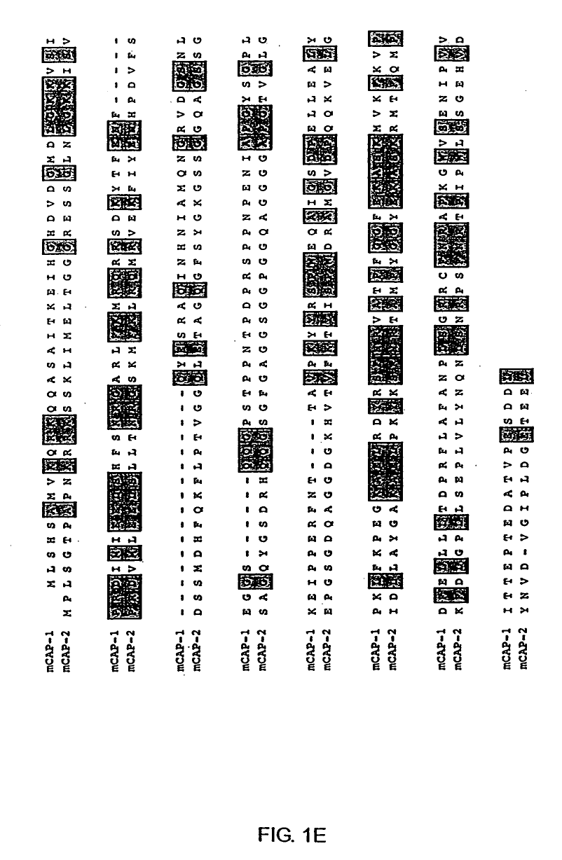

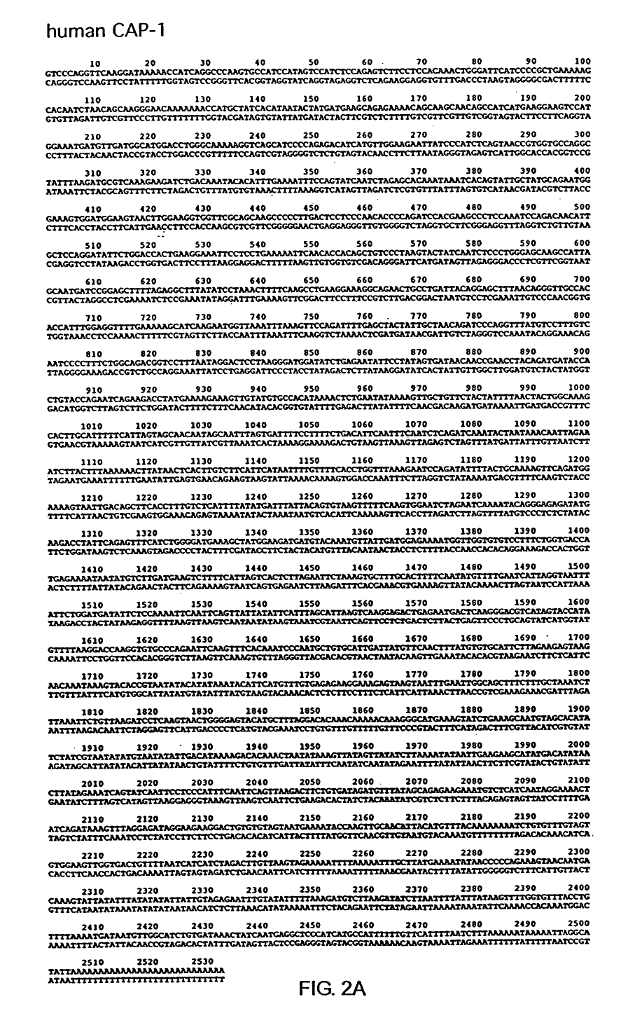

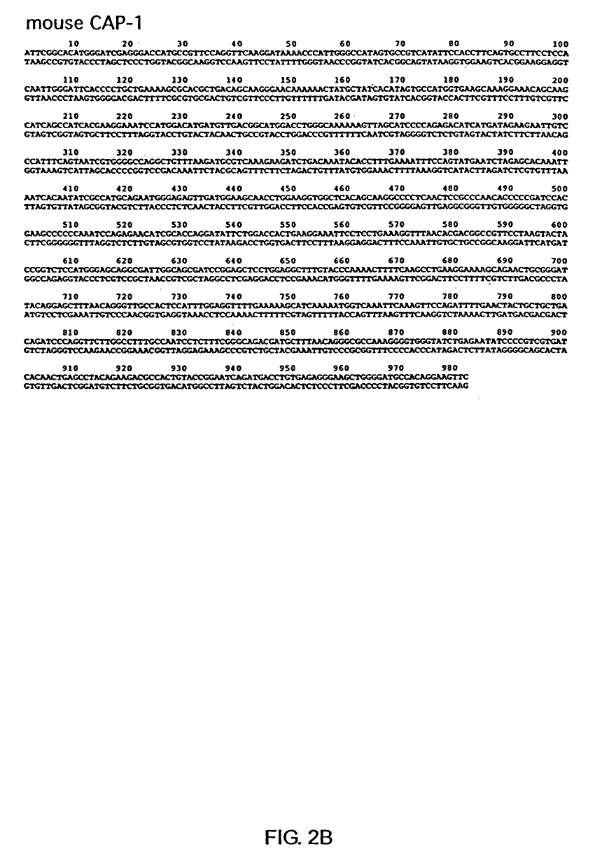

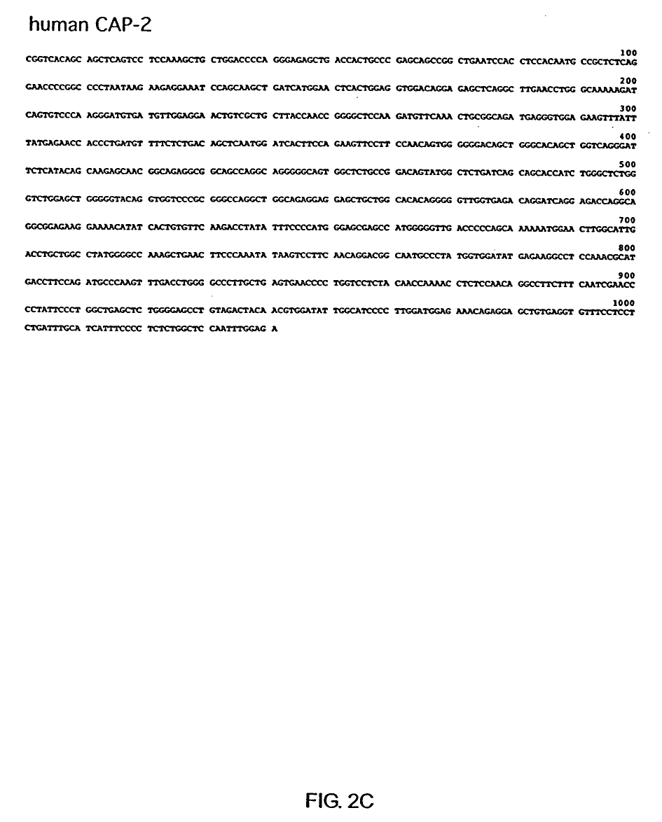

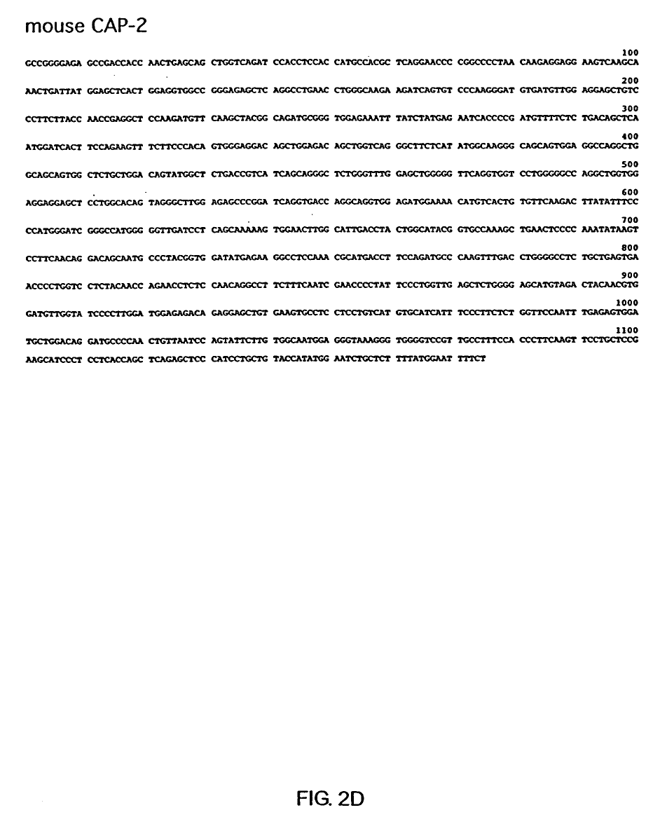

The deduced amino acid sequences of human calsarcin-1 (FIG. 1A), mouse calsarcin-1 (FIG. 1B), human calsarcin-2 (FIG. 1C) and mouse calsarcin-2 (FIG. 1D) are shown, along with an amino acid alignment of the mouse proteins (FIG. 1E). Also provided are DNA sequences for human calsarcin-1 (FIG. 2A), mouse calsarcin-1 (FIG. 2B), human calsarcin-2 (FIG. 2C) and mouse calsarcin-2 (FIG. 2D). Calsarcin-1 and -2 show the highest homology toward their amino- and carboxy-termini, whereas the intervening amino acids are less well conserved. BLATS searches with both proteins sequences did not reveal any significant homology to know proteins.

Calsarcin-2 was identified by searching the EST database (http://www.ncbi.nlm.nih.gov/dbEST/index.html) with the sequence of calsarcin-1. Three mouse calsarcin-2 ESTs were identified: GenBank accession numbers (AA036142, AW742494, W29466). Additionally, four human calsarcin-2 ESTs were identified: GenBank accession numbers (AW964108, AA197193, AW000988, AA176945). The mouse calsarcin-2 ESTs are as follows: GenBank No. AA036142; GenBank No. AW742494; and GenBank No. W29466. The human calsarcin-2 ESTs are as follows: GenBank No. AW964108; GenBank No. AA197193; GenBank No. AW000988; and GenBank No. AA176945.

Calsarcin-3 was discovered “in silico” by comparing calsarcin 1 and calsarcin 2 sequences with the database. Human genomic DNA (AC 008453.3; public not Celera database) containing several homologous sequences was confirmed to be exons of calsarcin-3. Primers were designed and a human skeletal muscle library was screened for the full-length cDNA for human calsarcin-3 (FIG. 5). Similarly, a mouse skeletal library was screened and several independent and overlapping clones encoding for mouse calscarcin-3 were identified.

Two hybrid experimental reagents and design are discussed in detail elsewhere herein. In an alternative embodiment a yeast one hybrid system (Vidal and Legrain, 1999; Sieweke, 2000) is utilized to determine interaction of a calsarcin with a nucleic acid sequence, or a three-hybrid system is utilized to detect RNA-protein interactions in vivo (SenGupta et al, 1996).

In other embodiments, other methods well known in the art are utilized to identify proteins or peptides which interact with calcineurin. For instance, a labeled form of calcineurin is generated by standard means in the art, a pool of potential interacting candidates are exposed to the labeled calcineurin, and the resultant interactors are identified. Alternatively, an unlabeled form of calcineurin is exposed to labeled candidates, and the resultant labeled interactor candidate, following exposure to the unlabeled calcineurin, is characterized. In an alternative specific embodiment, an unlabeled form of calcineurin is exposed to ³⁵S-labeled proteins, via ³⁵S-labeled methionine, such as is present in a cellular extract. The labeled interactor candidate is isolated and identified, such as by Sanger sequencing. In another embodiment, immunoprecipitation is performed by means well known in the art wherein antibodies to calcineurin are incubated with a source of candidate interactors, and the antibodies act to isolate or “pull down” any gene product which interacts with the form of calcineurin to which the antibody is bound. A skilled artisan is aware that the methods described herein regarding protein-protein interactions analogously apply to any protein or polypeptide, including all calsarcins. Other methods to determine protein-protein interactions are well known in the art.

Thus, in addition to a two hybrid system, additional methods to analyze protein interactions include interaction trap, affinity purification, phage-based expression cloning (also referred to as interaction cloning), surface plasmon resonance, and coprecipitation, described in its immunological form elsewhere herein.

In an interaction trap, also referred to as an interactor hunt, a yeast strain contains two LexA operator-responsive reporters: a chromosomally integrated LEU2 gene and a plasmid-borned GALlpromoter-lacZ fusion gene. Additionally, the strain contains a constitutively expressed chimeric protein comprising the LexA DNA-binding domain and the protein of interest, which is unable to independently activate the reporter genes. An inducible yeast GAL1 promoter drives expression of an activation domain-fused cDNA library, which is introduced into the yeast. Plating the tranformed yeast on galactose containing media which also lacks leucine induces expression of the library. If interaction of the bait protein with a candidate target protein occurs, LEU2 is expressed and colony growth is permitted. Expression of the reporter gene is confirmed with plating on medium containing X-gal.

Affinity purification, also known as GST pulldown purification, utilizes proteins fused to glutathione-S-transferase (GST) bound to glutathione-agarose beads. Exposure of the beads to a candidate interactor protein, which may be labeled or purified, is followed by subsequent washing. The quantity of candidate interactor protein retained is determined by either subjecting the beads/bound proteins to SDS-polyacrylamide electrophoresis or eluting with glutathione or salt. Although in a specific embodiment the candidate interactor protein is known, this method may also be used to test a complex mixture of proteins, such as with a crude cellular lysate, if performed in conjunction with other techniques or reagents, such as using antibodies to the candidate interactor protein.

In interaction cloning, also referred to as expression cloning, a nucleic acid encoding a bait protein (protein of interest) and an appropriate expression library, such as from a heart or muscle tissue, is present in a bacteriophage expression vector, such as λgt11. In a specific embodiment, a fusion protein consists of bait protein and GST but also including a recognition site for cyclic adenosine 3′,5′-phophate (cAMP)-dependent protein kinase A (PKA) site between them. The cDNA is radioactively labeled with ³²P The bait fusion protein is enzymatically phorphorylated by PKA and (γ-³²P)ATP. The labeled probe is utilized to screen a α bacteriophage-derived cDNA expression library expressing β-galactosidase fusion proteins containing in-frame gene fusions. Fusion proteins are adsorbed onto nitrocellulose membranes following lyses of the cells by the phage and plaque formation. Interacting clones are visualized, such as with autoradiography.

Surface plasmon resonance (SPR) is utilized to determine interaction with specific potential interacting analytes, and thus is best used when specific proteins are suspected to interact with a protein of interest. In surface plasmon resonance, a protein is immobilized on a chip which is exposed to a continuously flowing buffer. When sample “plugs” containing potential binding analytes are sequentially flowed over the protein surfact, the flow of the buffer is interrupted. A sensing apparatus on a SPR device, such as a BIAcore instrument, detects changes in the angle of minimum reflectance from the interface that result upon association of the potential interacting analyte with the protein of interest. Therefore, visualization of the molecular interactions occurs in real time, as seen on a computer monitor.

### Example 3

### Expression of Nucleic Acids Encoding Calcineurin-Associated Proteins

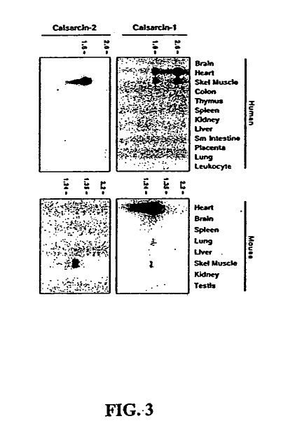

To determine which tissues calsarcin-1 and calsarcin-2 are expressed in, Northern analysis was performed. In FIG. 3, polyA+ RNA from the indicated mouse tissues was analyzed for expression of calsarcin-1 and calsarcin-2 transcripts by methods well known in the art. The data shows a highly striated, muscle-specific expression pattern for calsarcin-1 and -2. Calsarcin-1 is specifically expressed in the heart and skeletal muscle, with two mRNAs of 1.6 and 2.6 kb in human tissues, and only a single transcript of 1.3 kb in mouse. Faint expression of calsarcin-1 was also detected in mouse lung and liver. A 1.6 kb and 1.3 kb calsarcin-2 transcript was detected exclusively in adult human and mouse skeletal muscle, respectively. The relative difference in expression level of calsarcin-1 between human and mouse skeletal muscle may reflect differences in slow-versus fast-twitch fiber composition.

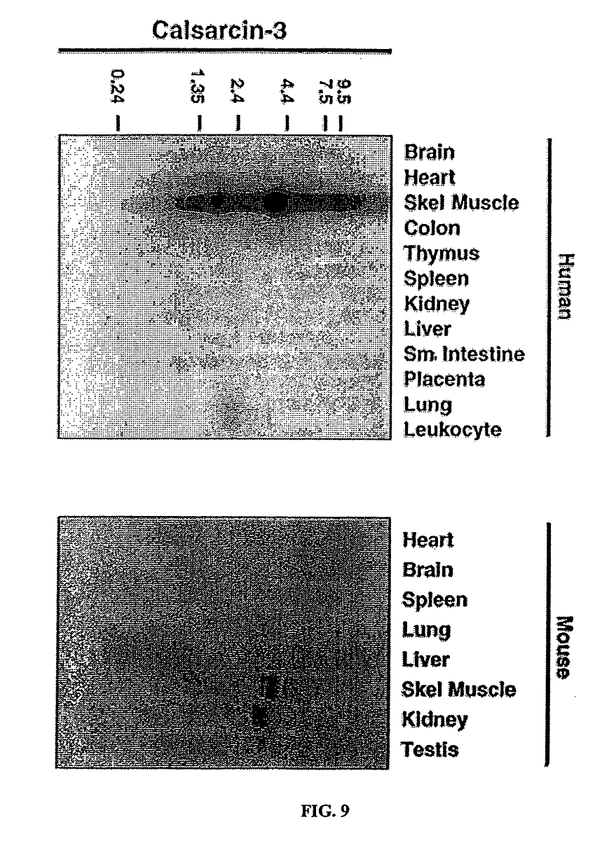

In a specific embodiment, the expression pattern of calsarcin-3 is determined by similar methods (FIG. 9). Methods to analyze RNA are well known. Briefly, RNA is isolated from a tissue of interest using standard techniques and is subsequently fractionated on an agarose gel, transferred to a membrane, and cross-linked to the membrane. A labeled probe is hybridized to the membrane, and the hybridization is detected.

Given the important role of calcineurin in regulating skeletal muscle hypertrophy and slow fiber gene expression, it is likely that calsarcin-2 plays an important role in regulating the functions of skeletal muscle.

### Example 4

### Localization of Expression of Nucleic Acids Encoding Calcineurin-Associated Proteins and Fiber Type Specificity of Calsarcin-1 and -2 in Skeletal Muscle

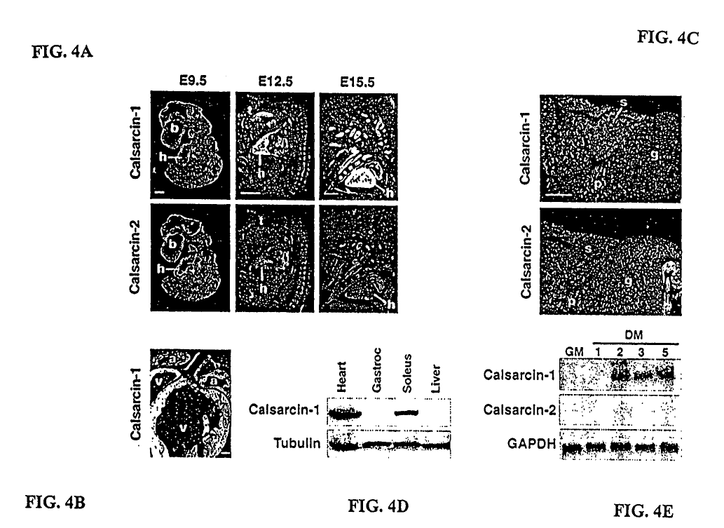

To characterize temporal and spatial patterns of expression of calsarcin-1, in situ hybridizations were performed. At embryonic day (E) 9.5, relatively weak expression of calsarcin-1 was observed in the heart, whereas at E12.5 and E15.5, intense signals were detected in both cardiac and skeletal muscle tissue. In contrast, adjacent sections from the same embryo probed with calsarcin-2 displayed significant cardiac expression at E9.5, which was still detectable at E12.5. Low level expression of calsarcin-2 in skeletal muscle of the tongue was also visible at this stage. At E15.5, cardiac expression of calsarcin-2 was downregulated and was only weakley detected in the atria, whereas skeletal muscle expression became more robust. Expression of calsarcin-1 in all cardiac chambers persisted through adulthood (FIG. 4B). Thus, calsarcin-1 is expressed in all striated muscle tissues throughout development, whereas calsarcin-2 is transiently expressed in the heart during early embryogenesis and later becomes restricted to skeletal muscle.

A skilled artisan is aware of standard methods to determine expression of a nucleic acid by in situ hybridization of tissues (Ausubel et al, 1994), such as by using fluorescence in situ hybridization (FISH). For in situ hybridization, a specific labeled nucleic acid probe is hybridized to a respective cellular nucleic acid, such as a RNA in a sample, such as tissue sections or individual cells. In specific embodiments, the samples were fixed for the appropriate time and dehydrated through a graded ethanol series. The samples were then impregnated in paraffin wax, cast into blocks and sectioned on a microtome. A specific labeled probe was prepared, such as with biotin, digoxigenin or with a fluorochrome-tagged deoxynucleotide. Next, the probe was hybrized to the sample. Hybridization conditions may vary depending on the nature of the labeled probe and the sample being tested. Following hybridization, in a specific embodiment, samples were washed for 15 min in 37 C 50% formamide/2×SSC, 15 min in 37 C₂×SSC and 15 min in room temperature 1×SSC. The slides were equilibrated for 5 min in 4×SSC at room temperature. The slides were drained and allowed to air dry. Next, a detection solution was added. After a 45 min incubation in the detection solution, the slides were washed. A counterstain, such as DAPI or propidium iodide staining solution was added to the slide. The slide was viewed using a fluorescence microscope.

In other embodiments, in situ hybridizations with other calsarcins are performed analogously. A skilled artisan is aware that, in an alternative embodiment, immunohistochemical localization of a polypeptide or protein is used to determine its localization subcellularly or to a particular cell type within tissues. In another embodiment, in situ hybridization and immunohistochemical localization are used in conjunction to determine location within a cell or tissue, thereby providing information regarding the nature of the function of the peptide or protein in question.

To determine whether calsarcins might exhibit fiber type-specificity of expression in skeletal muscle, we performed in situ hybridizations with sections of adult mouse hindlimb, using calsarcin-1 and -2 probes (FIG. 4C). Calsarcin-1 expression was localized to soleus and plantaris, which is comprises predominantly of slow-twitch fibers. In contrast, calsarcin-2 expression is enriched in gastrocnemius, which is primarily a fast-twitch muscle type.

Western blots of various-tissue extracts using calsarcin-1 antiserum revealed a single 32 kDa protein in heart and soleus (FIG. 4D). No expression was detected in liver or other non-muscle tissues. The calsarcin-1 antiserum did not recognize recombinant calsarcin-2 in extrasts derived from transfected Cos cells, indicating no significant cross-reactivity of the antiserum (data not shown). Only faint expression of calsarcin-1 protein could be detected in extract derived gastrocnemius (FIG. 4D), confirming the slow fiber-restricted expression of calcineurin-1.

Calsarcin-1 transcripts were upregulated during differentiation of the C2 skeletal muscle cell line, following transfer of proliferating myoblasts to differentiation medium (FIG. 3E). In contrast, calsarcin-2 expression was undetectable in C2 cells.

### Example 5

### Colocalization of Calsarcin-1 and α-actinin

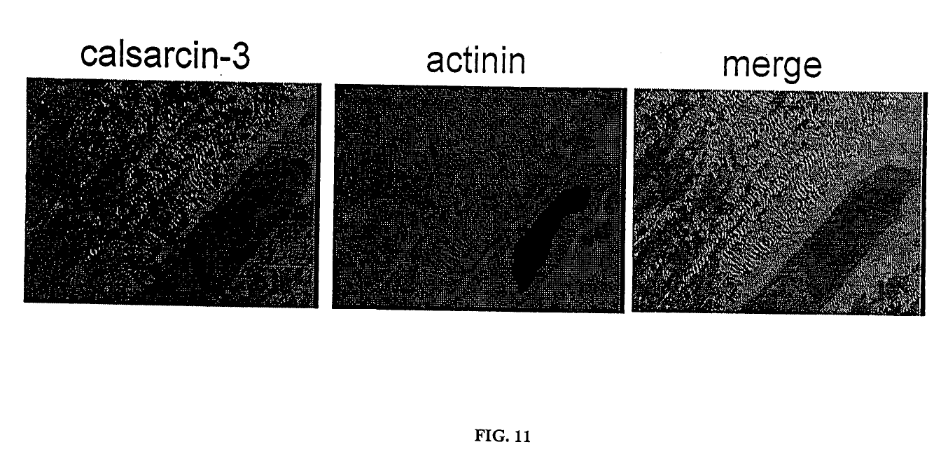

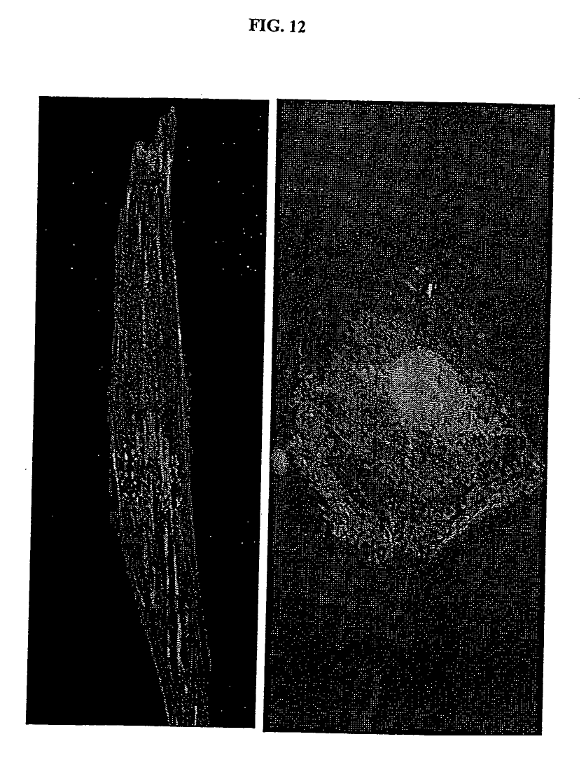

In light of two hybrid experiments demonstrating that calsarcin-1 and α-actinin interact, colocalization of the two gene products was tested. The subcellular localization of calsarcin-1 was determined by immunostaining of neonatal rat cardiomyocytes and cryosections of adult mouse heart and skeletal muscle. As shown in FIG. 5, calsarcin staining was localized to to the sarcomere of nenonatal cardiomyocytes and overlapped with α-actinin staining, which specifically marks the z-line. A similar staining pattern was observed in sections of adult mouse heart and skeletal muscle. Interestingly, calcineurin, detected with an antibody directed against amino acids 247-449 (Transductions Laboratories), was also colocalized to the z-line, indicating a muscle specific subcellular localization of the enzyme. The latter finding was confirmed by a second can antibody (Sigma). CnA staining was also detected in the nucleus, suggesting that calcineurin is also localized to other subcellular regions. In another embodiment, analagous experiments are performed with calsarcin-2 or calsarcin-3 antibodies to test for colocalization with a polypeptide such as α-actinin (FIG. 11). Furthermore, overexpression of calsarcin-1 in C2C12 myoblast cells, resulted in early (after one day of differentiation) and enhanced sarcomere formation. (FIG. 12)

### Example 6

### Identification of Proteins which Interact with Calcineurin-Associated Proteins

To further understand the functions of calsarcin-1, it was used as bait in a two-hybrid screen of muscle cDNA libraries, analogous to methods described in Example 1 and elsewhere herein. From this screen, numerous independent cDNAs encoding portions of α-actinin were identified. In a specific embodiment, calsarcin-2 and/or calsarcin-3 are used as bait in similar methods to detect calsarcin-2 or calsarcin-3-interacting polypeptides, respectively. α-actinin is normally associated with the Z-band of the sarcomere.

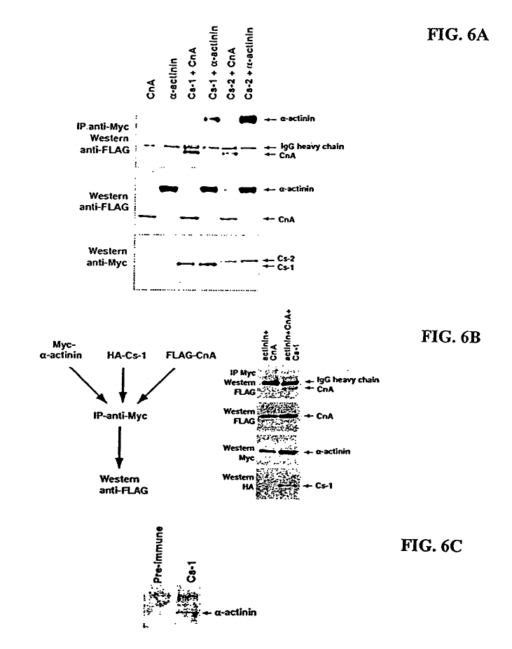

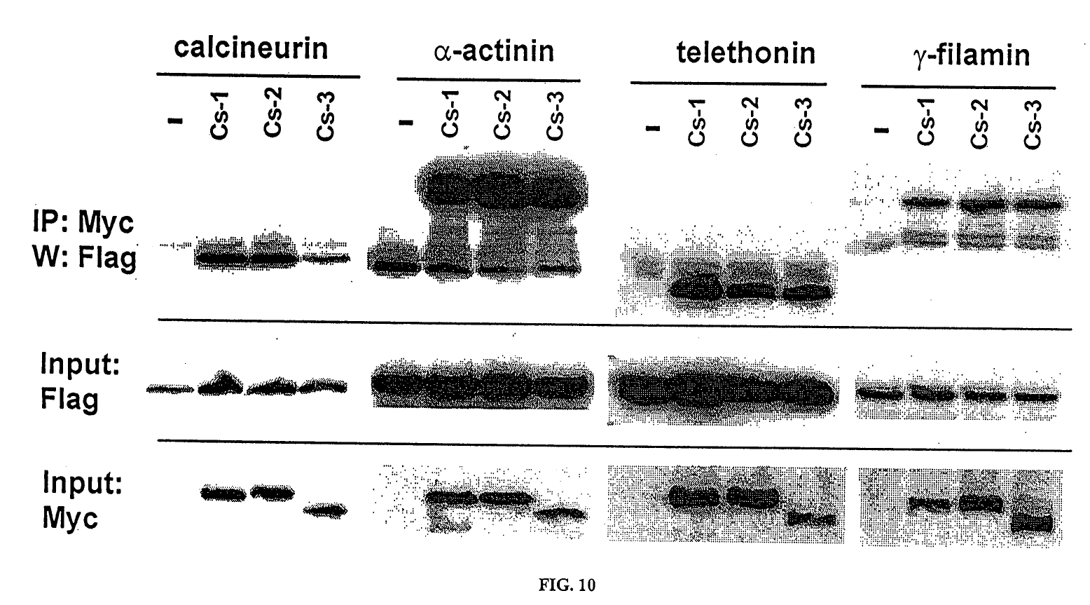

Association of calsarcin-1 and -2 with α-actinin was further tested by coimmunoprecipitation of epitope-tagged proteins in transfected Cos cells and of the native proteins from neonatal cardiomyocytes. As shown in FIG. 6A, C-terminal Myc-tagged calsarcin-1 immunoprecipitated FLAG-tagged CnA and α-actinin. Catalytic activity of calcineurin is not required for the calsarcin interaction as demonstrated by the ability of calsarcin-1 to immunoprecipitate a catalytically inactive CnA mutant. Using a triple-immunoprecipitation approach with Myc-tagged α-actinin, HA-tagged calsarcin and FLAG-tagged calcineurin (FIG. 6B), we demonstrated that CnA could only be precipitated by Myc-α-actinin in the presence of calsarcin-1, indicating a trimeric complex. In addition, α-actinin could also be coimmunoprecipitated with native calsarcin-1 from cardiomyocyte extracts (FIG. 6C). Furthermore, calsarcins 1-3 coimmunoprecipitated with the sarcomeric protein telethonin as demonstrated in FIG. 10. Telethonin is a disease gene involved in limb-girdle muscular dystrophy and may play a role in the stretch-response of striated muscle both in cardiac and skeletal muscle.

### Example 7

### Identification of Domains for Interaction of Calsarcin-1 with α-actinin

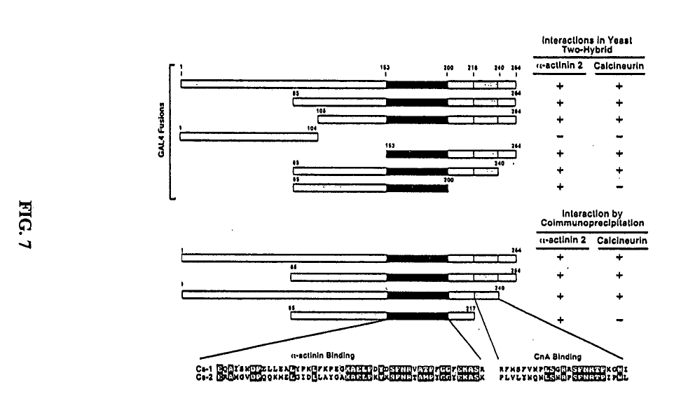

N- and C-terminal truncations of calsarcin-1 were used to characterize the CnA and α-actinin interaction domains. Yeast two-hybrid assays and complementary immunoprecipitation experiments revealed that amino acids 153-200 are necessary for interaction of calsarcin with α-actinin-2 (FIG. 7). Twenty-five residues within this region are highly conserved between mouse and human calsarcin-1 and -2, suggesting that this might constitute the minimal interaction domain. Since a motif between amino acids 245-250 resembles known calcineurin dockings sites on NFAT (PxIxIT) and MCIP (PxIxxIT), the inventors tested a C-terminal truncation lacking both those residues. However, calsarcin lacking these amino acids was still able to bind can, both by two-hybrid assay (GAL4-calsarcin 85-240) and coimmunoprecipitation (Myc-calsarcin 1-240). In contrast, a calsarcin-1 mutant lacking residues 217-264 was unable to bind CnA, implying that residues 217-240 are necessary for binding. Mapping of the interaction domain on CnA revealed that the calsarcin-interacting domain residues within the catalytic region, whereas the calsarcin-1 interacting domain of α-actinin maps to the second and third spectrin-like repeats.

In a specific embodiment, analogous experiments are performed with other calsarcins in identifying calsarcin domains for interaction with protein binding.

### Example 8

### Significance of Calcineurin-Associated Proteins in Cardiomyopathies and Muscular Dystrophies

Based on their interactions and colocalization in vivo, it also is proposed herein that calsarcin-1 links calcineurin to the Z-band where it can sense changes in calcium signaling in the myocyte and potentially transduce a hypertrophic signal (FIG. 8). Calsarcin-1, and/or other calsarcin proteins, such as calsarcin-2 or calsarcin-3, may also play structural and/or mechanosensory roles in cardiac and skeletal myocytes through modulation of the Z-band and its association with other proteins in the cell. The Z-band has been shown to play important roles in regulating muscle cell structure and function. Thus, calsarcin-1 is likely to be intimately involved in these processes and is a strong candidate for a gene involved in human cardiomyopathies and muscular dystrophies.

### REFERENCES

The following references, to the extent that they provide exemplary procedural or other details supplementary to those set forth herein, are specifically incorporated herein by reference.

U.S. Pat. No. 4,196,265, issued Apr. 1, 1980.

U.S. Pat. No. 4,554,101, issued Nov. 19, 1985.

U.S. Pat. No. 4,683,195, issued Jul. 28, 1987.

U.S. Pat. No. 4,683,202, issued, issued Jul. 28, 1987.

U.S. Pat. No. 4,800,159, issued Jan. 24, 1989.

U.S. Pat. No. 4,873,191, issued issued Oct. 10, 1989.

U.S. Pat. No. 4,883,750, issued Nov. 28, 1989.

U.S. Pat. No. 5,279,721, issued Jan. 18, 1994.

U.S. Pat. No. 5,354,855, issued Oct. 11, 1994.

U.S. Pat. No. 5,776,689, issued Jul. 7, 1998.

EPO 0273085, issued Jul. 6, 1988.

WO 84/03564, issued Sep. 13, 1984.

WO 90/07641 filed Dec. 21, 1990.

Ausubel, F. M, Brent, R, Kingston, R. E, Moore, D. D, Seidman, J. G, Smith, J. A, Struhl, K. (eds.) Current Protocols in Molecular Biology, John Wiley and Sons, Inc. (1994).

Barany, G. and Merrifield, R. B. A chromatographic method for the quantitative analysis of the deprotection of dithiasuccinoyl (Dts) amino acids. Anal. Biochem. May;95(1):160-70 (1979).

Barnes K V, Cheng G, Dawson M M, Menick D R. Cloning of cardiac, kidney, and brain promoters of the feline ncx1 gene. J Biol. Chem. April 25;272(17):11510-7 (1997).

Baron M D, Davison M D, Jones P, Patel B, Critchley D R. Isolation and characterization of a cDNA encoding a chick alpha-actinin. J Biol. Chem. Feb 25;262(6):2558-61 (1987).

Barski O A, Gabbay K H, Bohren K M. Characterization of the human aldehyde reductase gene and promoter. Genomics 60(2):188-98 (1999).

Beggs A H, Byers T J, Knoll J H, Boyce F M, Bruns G A, Kunkel L M. Cloning and characterization of two human skeletal muscle alpha-actinin genes located on chromosomes 1 and 11. J Biol. Chem. May 5;267(13):9281-8 (1992).

Bennett J P, Zaner K S, Stossel T P. Isolation and some properties of macrophage alpha-actinin: evidence that it is not an actin gelling protein. Biochemistry. October 9;23(21):5081-6 (1984).

Bhavsar P K, Brand N J, Yacoub N H, Barton P J R. Isolation and characterization of the human cardiac troponin I gene (TNNI3). Genomics. Jul 1;35(1):11-23 (1996).

Burridge K, Feramisco J R. Microinjection and localization of a 130K protein in living fibroblasts: a relationship to actin and fibronectin. Cell. March;19(3):587-95 (1980).

Capaldi R A, Bell R L, Branchek T. Changes in order of migration of polypeptides in complex III and cytochrome C oxidase under different conditions of SDS polyacrylamide gel electrophoresis. Biochem Biophys Res Commun. Jan 24;74(2):425-33 (1977).

Chin E R, Olson E N, Richardson J A, Yang Q, Humphries C, Shelton J M, Wu H, Zhu W, Bassel-Duby R, Williams R S. A calcineurin-dependent transcriptional pathway controls skeletal muscle fiber type. Genes Dev. August 15;12(16):2499-509 (1998).

Crabtree G R. Generic signals and specific outcomes: signaling through Ca2+, calcineurin, and NF-AT. Cell. March 5;96(5):611-4 (1999).

de Arruda M V, Watson S, Lin C S, Leavitt J, Matsudaira P. Fimbrin is a homologue of the cytoplasmic phosphoprotein plastin and has domains homologous with calmodulin and actin gelation proteins. J. Cell Biol. September;111(3):1069-79 (1990).

Ding B, Price R L, Borg T K, Weinberg E O, Halloran P F, Lorell B H. Pressure overload induces severe hypertrophy in mice treated with cyclosporine, an inhibitor of calcineurin. Circ Res. April 2;84(6):729-34 (1999).

Duhaiman A S, Bamburg J R. Isolation of brain alpha-actinin. Its characterization and a comparison of its properties with those of muscle alpha-actinins. Biochemistry. April 10;23(8):1600-8 (1984).

Dunn S E, Burns J L, Michel R N. Calcineurin is required for skeletal muscle hypertrophy. J Biol. Chem. July 30;274(31):21908-12 (1999).

Fashena S J, Serebriiskii I, Golemis E A. The continued evolution of two-hybrid screening approaches in yeast: how to outwit different preys with different baits. Gene 2000 May 30;250(1-2):1-14

Fields, S & Song, O. Nature 340:245-247 (1989).

Forster A C, Symons R H. Self-cleavage of virusoid RNA is performed by the proposed 55-nucleotide active site. Cell. July 3;50(1):9-16 (1987).

Franz W M, Brem G, Katus H A, Klingel K, Hofschneider P H, Kandolf R. Characterization of a cardiac-selective and developmentally upregulated promoter in transgenic mice. Cardioscience. December;5(4):235-43 (1994).

Fyrberg C, Ketchum A, Ball E, Fyrberg E. Characterization of lethal Drosophila melanogaster alpha-actinin mutants. Biochem Genet. October;36(9-10):299-310 (1998).

Gopal-Srivastava R, Haynes J I 2nd, Piatigorsky J. Regulation of the murine alpha B-crystallin/small heat shock protein gene in cardiac muscle. Mol Cell Biol. December;15(12):7081-90 (1995).

Graef I A, Mermelstein P G, Stankunas K, Neilson J R, Deisseroth K, Tsien R W, Crabtree G R. L-type calcium channels and GSK-3 regulate the activity of NF-ATc4 in hippocampal neurons. Nature. October 14;401(6754):703-8 (1999).

Harlow, E. Using antibodies: a laboratory manual. Cold Spring Harbor Laboratory Press, 1999, 495 pp.

Hill J A, Karimi M, Kutschke W, Davisson R L, Zimmerman K, Wang Z, Kerber R E, Weiss R M. Cardiac hypertrophy is not a required compensatory response to short-term pressure overload. Circulation. 2000 Jun. 20;101(24):2863-9.

Ho I C, Hodge M R, Rooney J W, Glimcher L H. The proto-oncogene c-maf is responsible for tissue-specific expression of interleukin-4. Cell. June 28;85(7):973-83 (1996).

Izumo S, Aoki H. Calcineurin—the missing link in cardiac hypertrophy. Nat Med. June;4(6):661-2 (1998).

James P, Halladay J, Craig E A Genomic libraries and a host strain designed for highly efficient two-hybrid selection in yeast. Genetics 1996 December;144(4):1425-36.

Kashishian A, Howard M, Loh C, Gallatin W M, Hoekstra M F, Lai Y. AKAP79 inhibits calcineurin through a site distinct from the immunophilin-binding region. J Biol. Chem. October 16;273(42):27412-9 (1998).

Kelly R, Alonso S. Tajbakhsh S. Cossu G, Buckingham M. Myosin light chain 3F regulatory sequences confer regionalized cardiac and skeletal muscle expression in transgenic mice. J Cell Biol. April;129(2):383-96 (1995).

Kimura S, Abe K, Suzuki M, Ogawa M, Yoshioka K, Kaname T, Miike T, Yamamura K. A 900 bp genomic region from the mouse dystrophin promoter directs lacZ reporter expression only to the right heart of transgenic mice. Dev Growth Differ. June;39(3):257-65 (1997).

Klee C B, Ren H, Wang X. Regulation of the calmodulin-stimulated protein phosphatase, calcineurin. J Biol. Chem. May 29;273(22):13367-70 (1998).

Kyte J, Doolittle R F. A simple method for displaying the hydropathic character of a protein. J Mol. Biol. May 5;157(1):105-32 (1982).

Lai M M, Burnett P E, Wolosker H, Blackshaw S, Snyder S H. Cain, a novel physiologic protein inhibitor of calcineurin. J Biol. Chem. July 17;273(29):18325-31 (1998).

Landon F, Gache Y, Touitou H, Olomucki A. Properties of two isoforms of human blood platelet alpha-actinin. Eur J Biochem. Dec 2;153(2):231-7 (1985).

LaPointe M C, Wu G, Garami M, Yang X P, Gardner D G. Tissue-specific expression of the human brain natriuretic peptide gene in cardiac myocytes. Hypertension. March;27(3 Pt 2):715-22 (1996).

Lim H W, De Windt L J, Steinberg L, Taigen T, Witt S A, Kimball T R, Molkentin J D. Calcineurin expression, activation, and function in cardiac pressure-overload hypertrophy. Circulation. 2000 May 23;101(20):2431-7.

Lim H W, De Windt L J, Mante J, Kimball T R, Witt S A, Sussman M A, Molkentin J D. Reversal of cardiac hypertrophy in transgenic disease models by calcineurin inhibition. J Mol Cell Cardiol. 2000 April;32(4):697-709.

Liu Y C, Elly C, Langdon W Y, Altman A. Ras-dependent, Ca2+-stimulated activation of nuclear factor of activated T cells by a constitutively active Cb1 mutant in T cells. J Biol. Chem. January 3;272(1):168-73 (1997).

Lu, J, Richardson, J, Gan, L, Olson, E. Mech Dev 73:23-32 (1998).

Luther P K. Three-dimensional reconstruction of a simple Z-band in fish muscle. J. Cell Biol. June;113(5):1043-55 (1991).

Macejak D G, Samow P. Internal initiation of translation mediated by the 5′ leader of a cellular mRNA. Nature. September 5;353(6339):90-4 (1991).

Mao Z, Wiedmann M. Calcineurin enhances MEF2 DNA binding activity in calcium-dependent survival of cerebellar granule neurons. J Biol. Chem. October 22;274(43):31102-7 (1999).

Mao Z, Bonni A, Xia F, Nadal-Vicens M, Greenberg M E. Neuronal activity-dependent cell survival mediated by transcription factor MEF2. Science. Oct 22;286(5440):785-90 (1999).

Marban E, Kitakaze M, Kusuoka H, Porterfield J K, Yue D T, Chacko V P. Intracellular free calcium concentration measured with ¹⁹F NMR spectroscopy in intact ferret hearts. Proc Natl Acad Sci USA. August;84(16):6005-9 (1987).

Merrifield B. Solid phase synthesis. Science. April 18;232(4748):341-7 (1986).

Mesaeli N, Nakamura K, Zvaritch E, Dickie P, Dziak E, Krause K H, Opas M, MacLennan D H, Michalak M. Calreticulin is essential for cardiac development. J. Cell Biol. March 8;144(5):857-68 (1999).

Michel F, Westhof E. Modelling of the three-dimensional architecture of group I catalytic introns based on comparative sequence analysis. J Mol. Biol. December 5;216(3):585-610 (1990).

Molkentin J D, Lu J R, Antos C L, Markham B, Richardson J, Robbins J, Grant S R, Olson E N. A calcineurin-dependent transcriptional pathway for cardiac hypertrophy. Cell. April 17;93(2):215-28 (1998).

Musaro A, McCullagh K J, Naya F J, Olson E N, Rosenthal N. IGF-1 induces skeletal myocyte hypertrophy through calcineurin in association with GATA-2 and NF-ATc1. Nature. August 5;400(6744):581-5 (1999).

Olson E N, Molkentin J D. Prevention of cardiac hypertrophy by calcineurin inhibition: hope or hype? Circ Res. 1999 April 2;84(6):623-32 (1999).

Parr T, Waites G T, Patel B, Millake D B, Critchley D R. A chick skeletal-muscle alpha-actinin gene gives rise to two alternatively spliced isoforms which differ in the EF-hand Ca(2+)-binding domain. Eur J. Biochem. December 15;210(3):801-9 (1992).

Pelletier J, Sonenberg N. Internal initiation of translation of eukaryotic mRNA directed by a sequence derived from poliovirus RNA. Nature. 1988 Jul. 28;334(6180):320-5.

Rao A, Luo C, Hogan P G. Transcription factors of the NFAT family: regulation and function. Annu Rev Immunol.;15:707-47 (1997).

Reinhold-Hurek B, Shub D A. Self-splicing introns in tRNA genes of widely divergent bacteria. Nature. May 14;357(6374):173-6 (1992).

Ritchie M E. Characterization of human B creatine kinase gene regulation in the heart in vitro and in vivo. J Biol. Chem. October 11;271(41):25485-91 (1996).

Sambrook, J, Fritsch, E. F, and Maniatis, T. Molecular Cloning: A Laboratory Manual. Cold Spring Harbor Laboratory (11989).

Schroeter J P, Bretaudiere J P, Sass R L, Goldstein M A. Three-dimensional structure of the Z band in a normal mammalian skeletal muscle. J. Cell Biol. May; 133(3):571-83 (1996).

Seidman C E, Seidman J G. Molecular genetic studies of familial hypertrophic cardiomyopathy. Basic Res Cardiol.;93 Suppl 3:13-6 (1998).

Semsarian C, Wu M J, Ju Y K, Marciniec T, Yeoh T, Allen D G, Harvey R P, Graham R M, Skeletal muscle hypertrophy is mediated by a Ca2+-dependent calcineurin signalling pathway. Nature. August 5;400(6744): 576-81 (1999).

SenGupta D J, Zhang B, Kraemer B, Pochart P, Fields S, Wickens M. A three-hybrid system to detect RNA-protein interactions in vivo. Proc Nat'l Acad Sci USA. 1996 August 6;93(16):8496-501.

Shibasaki, F, Price, E, Milan, D. and McKeon, F. Nature 1996 382:370-373.

Shimoyama M, Hayashi D, Takimoto E, Zou Y, Oka T, Uozumi H, Kudoh S, Shibasaki F, Yazaki Y, Nagai R, Komuro I. Calcineurin plays a critical role in pressure overload-induced cardiac hypertrophy. Circulation. 1999 December 14; 100(24):2449-54.

Sieweke M. Detection of transcription factor partners with a yeast one hybrid screen. Methods Mol Biol 2000;130:59-77.

Spencer, J, Eliazer, S, Ilaria, R, Richardson, J, Olson, E. J Cell Biol 2000 150:771-784.

Squire J. Muscle regulation: a decade of the steric blocking model. Nature. June 25;291(5817):614-5 (1981).

Sussman M A, Lim H W, Gude N, Taigen T, Olson E N, Robbins J, Colbert M C, Gualberto A, Wieczorek D F, Molkentin J D. Prevention of cardiac hypertrophy in mice by calcineurin inhibition. Science. 1998 September 11;281(5383):1690-3.

Taigen T, De Windt L J, Lim H W, Molkentin J D. Targeted inhibition of calcineurin prevents agonist-induced cardiomyocyte hypertrophy. Proc Nat'l Acad Sci USA. 2000 February 1;97(3):1196-201.

Timmerman L A, Clipstone N A, Ho S N, Northrop J P, Crabtree G R. Rapid shuttling of NF-AT in discrimination of Ca2+ signals and immunosuppression. Nature. October 31;383(6603):837-40 (1996).

Vidal M, Legrain P. Yeast forward and reverse ‘n’-hybrid systems. Nucleic Acids Res 1999 February 15;27(4):919-29.

Vigoreaux J O. The muscle Z band: lessons in stress management. J Muscle Res Cell Motil. June;15(3):237-55 (1994).

Wang H G, Pathan N, Ethell I M, Krajewski S, Yamaguchi Y, Shibasaki F, McKeon F, Bobo T, Franke T F, Reed J C. Ca2+-induced apoptosis through calcineurin dephosphorylation of BAD. Science. April 9;284(5412):339-43 (1999).

Yang M, Wu Z, Fields S. Protein-peptide interactions analyzed with the yeast two-hybrid system. Nucleic Acids Res 1995 April 11;23(7): 1152-6.

Youn H D, Sun L, Prywes R, Liu J O. Apoptosis of T cells mediated by Ca2+-induced release of the transcription factor MEF2. Science. October 22;286(5440):790-3 (1999).

Zhang W, Kowal R C, Rusnak F, Sikkink R A, Olson E N, Victor R G. Failure of calcineurin inhibitors to prevent pressure-overload left ventricular hypertrophy in rats. Circ Res. April 2;84(6):722-8 (1999).

Zhuo M, Zhang W, Son H, Mansuy I, Sobel R A, Seidman J, Kandel E R. A selective role of calcineurin aalpha in synaptic depotentiation in hippocampus. Proc Nat'l Acad Sci USA. April 13;96(8):4650-5 (1999).

Ziober B L, Kramer R H. Identification and characterization of the cell type-specific and developmentally regulated alpha7 integrin gene promoter. J Biol. Chem. September 13;271(37):22915-22 (1996).

What is claimed is:

1. An isolated and purified polypeptide comprising SEQ ID NO:2.

2. An isolated and purified polypeptide consisting of SEQ ID NO:2.

3. An isolated and purified polypeptide comprising SEQ ID NO:6.

4. An isolated and purified polypeptide consisting of SEQ ID NO:6.

5. An isolated and purified polypeptide comprising SEQ ID NO:10.

6. An isolated and purified polypeptide consisting of SEQ ID NO:10.

7. The isolated and purified polypeptide of claim 1, further comprising an additional coding region.

8. The isolated and purified polypeptide of claim 3, further comprising an additional coding region.

9. The isolated and purified polypeptide of claim 5, further comprising an additional coding region.

10. An isolated and purified nucleic acid comprising a nucleic acid segment encoding SEQ ID NO:2.

11. The isolated and purified nucleic acid segment of claim 10, further comprising a promoter active in eukaryotic cells.

12. The isolated and purified nucleic acid segment of claim 10, wherein said nucleic acid further comprises a recombinant vector.

13. An isolated and purified nucleic acid comprising a nucleic acid segment encoding SEQ ID NO:6.

14. The isolated and purified nucleic acid segment of claim 13, further comprising a promoter active in eukaryotic cells.

15. The isolated and purified nucleic acid segment of claim 13, wherein said nucleic acid further comprises a recombinant vector.

16. An isolated and purified nucleic acid comprising a nucleic acid segment encoding SEQ ID NO:10.

17. The isolated and purified nucleic acid segment of claim 16, further comprising a promoter active in eukaryotic cells.

18. The isolated and purified nucleic acid segment of claim 16, wherein said nucleic acid further comprises a recombinant vector.

19. An isolated and purified nucleic acid segment, wherein said nucleic acid segment encodes a fusion polypeptide comprising SEQ ID NO:2.

20. An isolated and purified nucleic acid segment, wherein said nucleic acid segment encodes a fusion polypeptide comprising SEQ ID NO:6.

21. An isolated and purified nucleic acid segment, wherein said nucleic acid segment encodes a fusion polypeptide comprising SEQ ID NO:10.

22. A knockout non-human animal comprising a defective allele of a nucleic acid encoding a calcineurin associated sarcomeric protein (calsarcin).

23. The animal of claim 22, further comprising two defective alleles of a nucleic acid encoding a calsarcin.

24. The animal of claim 22, wherein said animal is a mouse.

25. A transgenic non-human animal comprising an expression cassette, wherein said cassette comprises a nucleic acid encoding a calsarcin polypeptide under the control of a promoter active in eukaryotic cells.

26. The animal of claim 25, wherein said promoter is constitutive.

27. The animal of claim 25, wherein said promoter is tissue specific.

28. The animal of claim 25, wherein said promoter is inducible.

29. The animal of claim 25, wherein said animal is a mouse.

30. A monoclonal antibody that binds immunologically to a polypeptide comprising SEQ ID NO:2, or an antigenic fragment thereof.

31. A polyclonal antisera, antibodies of which bind immunologically to a polypeptide comprising SEQ ID NO:2, or an antigenic fragment thereof.

32. A monoclonal antibody that binds immunologically to a polypeptide comprising SEQ ID NO:6, or an antigenic fragment thereof.

33. A polyclonal antisera, antibodies of which bind immunologically to a polypeptide comprising SEQ ID NO:6, or an antigenic fragment thereof.

34. A monoclonal antibody that binds immunologically to a polypeptide comprising SEQ ID NO:10, or an antigenic fragment thereof.

35. A polyclonal antisera, antibodies of which bind immunologically to a polypeptide comprising SEQ ID NO:10, or an antigenic fragment thereof.

36. A method of modulating calcineurin activity in an animal comprising the step of administering to said organism a calsarcin polypeptide, or a calcineurin-binding fragment thereof.

37. A method of modulating calcineurin activity in an animal comprising the step of administering to said organism a dominant-negative form of a calsarcin polypeptide, or a calcineurin-binding fragment thereof.

38. A method of modulating calcineurin activity in an animal comprising the step of administering to said animal a nucleic acid which encodes a calsarcin polypeptide, or a calcineurin-binding fragment thereof, said nucleic acid under the control of a promoter operable in cells of said animal.

39. The method of claim 38, wherein said promoter is a constitutive promoter.

40. The method of claim 38, wherein said promoter is a muscle-specific promoter.

41. The method of claim 40, wherein said muscle-specific promoter is myosin light chain-2 promoter, α actin promoter, troponin 1 promoter, Na⁺/Ca²⁺ exchanger promoter, dystrophin promoter, creatine kinase promoter, α7 integrin promoter, brain natriuretic peptide promoter, α B-crystallin/small heat shock protein promoter, α myosin heavy chain promoter or atrial natriuretic factor promoter.

42. The method of claim 38, wherein said nucleic acid comprises a viral vector.

43. A method of screening for a peptide which interacts with a calsarcin comprising the steps of:
(a) introducing into a cell:
a first nucleic acid comprising a DNA segment encoding a test peptide, wherein said test peptide is fused to a DNA binding domain; and
a second nucleic acid comprising a DNA segment encoding at least a part of calsarcin, wherein said at least part of calsarcin is fused to a DNA activation domain; and
(b) assaying for an interaction between said test peptide and said at least part of calsarcin by assaying for an interaction between said DNA binding domain and said DNA activation domain.

44. The method of claim 43, wherein said DNA binding domain and said DNA activation domain are selected from the group consisting of GAL4 and LexA.

45. A method of screening for a modulator of calsarcin binding to α-actinin comprising:
(a) providing a calsarcin and α-actinin;
(b) admixing the calsarcin and α-actinin in the presence of a candidate modulator;
(c) measuring calsarcin/α-actinin binding; and
(d) comparing the binding in step (c) with the binding of calsarcin and α-actinin in the absence of said candidate modulator,
whereby a difference in the binding of calsarcin and α-actinin in the presence of said candidate modulator, as compared to binding in the absence of said candidate modulator, identifies said candidate modulator as a modulator of calsarcin binding to α-actinin.

46. The method of claim 45, wherein calsarcin and α-actinin are part of a cell free system.

47. The method of claim 45, wherein calsarcin and α-actinin are located within an intact cell.

48. The method of claim 47, wherein said cell is a myocyte.

49. The method of claim 47, wherein said cell is a H9C2 cell, a C2C12 cell, a 3T3 cell, a 293 cell, a neonatal cardiomyocyte cell or a myotube cell.

50. The method of claim 47, wherein said intact cell is located in an animal.

51. The method of claim 45, wherein said modulator increases calsarcin binding to α-actinin.

52. The method of claim 45, wherein said modulator decreases calsarcin binding to α-actinin.

53. The method of claim 45, wherein either or both calsarcin and α-actinin are labeled.

54. The method of claim 53, wherein both calsarcin and α-actinin are labeled, one with a quenchable label and the other with a quenching agent.

55. The method of claim 53, wherein both calsarcin and α-actinin are labeled, but said labels are not detectable unless brought into proximity of each other.

56. The method of claim 45, wherein measuring comprises immunologic detection of calsarcin, α-actinin or both.

57. The method of claim 45, further comprising measuring binding of calsarcin and α-actinin in the absence of a modulator.

58. A method of screening for a modulator of calsarcin binding to calcineurin comprising:
(a) providing a calsarcin and calcineurin;
(b) admixing the calsarcin and calcineurin in the presence of a candidate modulator;
(c) measuring calsarcin/calcineurin binding; and
(d) comparing the binding in step (c) with the binding of calsarcin and calcineurin in the absence of said candidate modulator,
whereby a difference in the binding of calsarcin and calcineurin in the presence of said candidate modulator, as compared to binding in the absence of said candidate modulator, identifies said candidate modulator as a modulator of calsarcin binding to calcineurin.

59. The method of claim 58, wherein calsarcin and calcineurin are part of a cell free system.

60. The method of claim 58, wherein calsarcin and calcineurin are located within an intact cell.

61. The method of claim 60, wherein said cell is a myocyte.

62. The method of claim 60, wherein said cell is a H9C2 cell, a C2C12 cell, a 3T3 cell, a 293 cell, a neonatal cardiomyocyte cell or a myotube cell.

63. The method of claim 60, wherein said intact cell is located in an animal.

64. The method of claim 58, wherein said modulator increases calsarcin binding to calcineurin.

65. The method of claim 58, wherein said modulator decreases calsarcin binding to calcineurin.

66. The method of claim 58, wherein either or both calsarcin and calcineurin are labeled.

67. The method of claim 66, wherein both calsarcin and calcineurin are labeled, one with a quenchable label and the other with a quenching agent.

68. The method of claim 66, wherein both calsarcin and calcineurin are labeled, but said labels are not detectable unless brought into proximity of each other.

69. The method of claim 58 wherein measuring comprises immunologic detection of calsarcin, calcineurin or both.

70. The method of claim 58 further comprising measuring binding of calsarcin and calcineurin in the absence of a modulator.

71. A method of screening for a modulator of calsarcin binding to telethonin comprising:
(a) providing a calsarcin and telethonin;
(b) admixing the calsarcin and telethonin in the presence of a candidate modulator;
(c) measuring calsarcin/telethonin binding; and
(d) comparing the binding in step (c) with the binding of calsarcin and telethonin in the absence of said candidate modulator,
whereby a difference in the binding of calsarcin and telethonin in the presence of said candidate modulator, as compared to binding in the absence of said candidate modulator, identifies said candidate modulator as a modulator of calsarcin binding to telethonin.

72. The method of claim 71, wherein calsarcin and telethonin are part of a cell free system.

73. The method of claim 71, wherein calsarcin and telethonin are located within an intact cell.

74. The method of claim 73, wherein said cell is a myocyte.

75. The method of claim 73, wherein said cell is a H9C2 cell, a C2C12 cell, a 3T3 cell, a 293 cell, a neonatal cardiomyocyte cell or a myotube cell.

76. The method of claim 73, wherein said intact cell is located in an animal.

77. The method of claim 71, wherein said modulator increases calsarcin binding to telethonin.

78. The method of claim 71, wherein said modulator decreases calsarcin binding to telethonin.

79. The method of claim 71, wherein either or both calsarcin and telethonin are labeled.

80. The method of claim 79, wherein both calsarcin and telethonin are labeled, one with a quenchable label and the other with a quenching agent.

81. The method of claim 79, wherein both calsarcin and telethonin are labeled, but said labels are not detectable unless brought into proximity of each other.

82. The method of claim 71 wherein measuring comprises immunologic detection of calsarcin, telethonin or both.

83. The method of claim 71 further comprising measuring binding of calsarcin and telethonin in the absence of a modulator.

84. A method of treating cardiac hypertrophy, heart failure or Type II diabetes comprising the step of administering to an animal suffering therefrom a calsarcin polypeptide, or a calcineurin-binding fragment thereof, wherein said calsarcin polypeptide or fragment thereof inhibits calcineurin activity.

85. A method of treating cardiac hypertrophy, heart failure or Type II diabetes, comprising the step of administering to an animal suffering therefrom a nucleic acid encoding a calsarcin polypeptide or a calcineurin binding fragment thereof, under the control of a promoter active in cardiac tissue, wherein expression of said calsarcin polypeptide or fragment thereof inhibits calcineurin activity.

86. The method of claim 85, wherein said polypeptide is a dominant negative form of calsarcin.

87. The method of claim 85, further comprising treating said animal with a compound selected from the group consisting of an ionotrope, a beta blocker, an antiarrhythmic, a diuretic, a vasodilator, a hormone antagonist, an endothelin antagonist, an angiotensin type 2 antagonist and a cytokine inhibitor/blocker.

88. The method of claim 85 wherein said promoter is a constitutive promoter.

89. The method of claim 85 wherein said promoter is an inducible promoter.

90. A method of inhibiting calcineurin activation of gene transcription in a cell comprising providing to said cell a fusion protein comprising calsarcin, or a calcineurin-binding fragment thereof, fused to a targeting peptide that localizes said fusion protein to a subcellular region other than a subcellular region of normal function for said calcineurin.

91. The method of claim 90, wherein said targeting peptide comprises a geranylgeranyl group, a nuclear localization signal, a myristilation signal, and an endoplasmic reticulum signal peptide.

92. The method of claim 90, wherein said cell is located in an animal.

93. The method of claim 92 wherein said animal is a human.

94. The method of claim 93 further comprising treating said animal with a compound selected from the group consisting of an lonotrope, a beta blocker, an antiarrhythmic, a diuretic, a vasodilator, a hormone antagonist, an endothelin antagonist, an angiotensin type 2 antagonist and a cytokine inhibitor/blocker.

95. A method of identifying a peptide that binds calsarcin comprising the steps of:
(a) attaching a calsarcin polypeptide, or a fragment thereof, to a support;
(b) exposing said calsarcin polypeptide or fragment to a candidate peptide; and
(c) assaying for binding of said candidate peptide to said calsarcin polypeptide or fragment thereof.

96. The method of claim 95, wherein said support is selected from the group consisting of nitrocellulose, a column, or a gel.

97. A method of screening for a candidate substance for anti-cardiomyopic hypertrophy activity or anti-heart failure activity comprising the steps of:
(a) providing a cell lacking a functional calsarcin polypeptide;
(b) contacting said cell with said candidate substance; and
(c) determining the effect of said candidate substance on said cell.

98. The method of claim 94, wherein said cell is a muscle cell.

99. The method of claim 97, wherein said cell has a mutation in a regulatory region of calsarcin.

100. The method of claim 97, wherein said mutation is a deletion mutation, an insertion mutation, or a point mutation.

101. The method of claim 97 wherein said cell has a mutation in the coding region of calsarcin.

102. The method of claim 101, wherein said mutation is a deletion mutation, an insertion mutation, a frameshift mutation, a nonsense mutation, a missense mutation or a splicing mutation.

103. The method of claim 97, wherein said cell is contacted in vitro.

104. The method of claim 97, wherein said cell is contacted in vivo.

105. The method of claim 105, wherein said cell is located in a non-human transgenic animal.


SEQUENCE LISTING (12 sequences)

SEQ ID NO: 1 - DNA, 2531 bp, Homo sapiens
gtcccaggttcaaggataaaaaccatcaggcccaagtgccatccatagtccatctccagagtcttcctccacaaactgggattcatccccgctgaaaaagcacaatctaacagcaagggaacaaaaaaaccatgctatcacataatactatgatgaagcagagaaaacagcaagcaacagccatcatgaaggaagtccatggaaatgatgttgatggcatggacctgggcaaaaaggtcagcatccccagagacatcatgttggaagaattatcccatctcagtaaccgtggtgccaggctatttaagatgcgtcaaagaagatctgacaaatacacatttgaaaatttccagtatcaatctagagcacaaataaatcacagtattgctatgcagaatgggaaagtggatggaagtaacttggaaggtggttcgcagcaagcccccttgactcctcccaacaccccagatccacgaagccctccaaatccagacaacattgctccaggatattctggaccactgaaggaaattcctcctgaaaaattcaacaccacagctgtccctaagtactatcaatctccctgggagcaagccattagcaatgatccggagcttttagaggctttatatcctaaacttttcaagcctgaaggaaaggcagaactgcctgattacaggagctttaacagggttgccacaccatttggaggttttgaaaaagcatcaagaatggttaaatttaaagttccagattttgagctactattgctaacagatcccaggtttatgtcctttgtcaatcccctttctggcagacggtcctttaataggactcctaagggatggatatctgagaatattcctatagtgataacaaccgaacctacagatgataccactgtaccagaatcagaagacctatgaaaagaaagttgtatgtgccacataaaactctgaatataaaagttgctgttctactattttaactactggcaaagcacttgcatttttcattagtagcaacaatagcaatttagtgattttccttttctgacattcaatttcaatctcagatcaaatactaataaacaattagaaatcttactttaaaaaacttataactcacttgtcttcattcataattttgttttcacctggtttaaagaatccagatattttactgcaaaagttcagatggaaaagtaattgacagcttcacctttgtctcattttatatgatttattacagtgtaagtttttcaagtggaatctagaatcaaaatacagggagagatatgaagacctattcagagtttcatctggggatgaaagctatggaagatgatgtacaaatgttattgatggagaaaatggttggtgtgtcctttctggtgaccatgagaaaataatatgtcttgatgaagtcttttcattagtcactcttagaattctaaagtgctttgcacttttcaatatgttttgaatcattaggtaatttattctggatgatattctccaaaattcaattcagttattatattcatttagcattaagtcaaggagactgagaatgactcaagggacgtcatagtaccatagttttaaggaccaaggtgtgcccagaattcaagtttcacaaatcccaatgctgtgcattgattatgttcaactttatgtgtgcattcttagaagagtaagaacaaataaagtacaccgtaatatacatataaatacattcatgtttgtgagagaaggaaagagtaagtaatttgaattggcagctttctttgctaaatctttaaattctgttaagatcctcaagtaactggggagtacatgctttaggacacaaacaaaaacaaagggcatgaaagtatctgaaagcaatgtagcacatatctatcgtaatatatgtaatatattgacataaaagacacaaactaatataaagttatagttatatcttaaaatataattgaagaagcatatgacatataacttatagaaatcagtatcaattcctcccatttcaattcagttaagactctgtgatagatgtttatagcagagaagaaatgtctcatcaataggaaaactatcagataaagtttaggagataggaagaaggactgtgtgtagtaatgaaaataccaagttgcaacattacatgtttacaaaaaaaatctgtgtttgtagtgtggaagttggtgactgttttaatcatcatctagacttgttaagtagaaaaattttaaaaatttgcttatgaaaatataacccccagaaagtaacaatgacaaagtattatatttatatatattattgtagagaatttgtatatttttaaagatgtcttaagatatcttaattttatttataagttttggtgtttacctgttttaaaatgataatgttggcatctgtgataaactatcaatgaggctcccatcatgccattttttgttcattttaatctttaaaaaataaaaattaggcatattaaaaaaaaaaaaaaaaaaaaaaaaaaaa

SEQ ID NO: 2 - protein, 264 aa, Homo sapiens
MLSHNTMMKQRKQQATAIMKEVHGNDVDGMDLGKKVSIPRDIMLEELSHLSNRGARLFKMRQRRSDKYTFENFQYQSRAQINHSIAMQNGKVDGSNLEGGSQQAPLTPPNTPDPRSPPNPDNIAPGYSGPLKEIPPEKFNTTAVPKYYQSPWEQAISNDPELLEALYPKLFKPEGKAELPDYRSFNRVATPFGGFEKASRMVKFKVPDFELLLLTDPRFMSFVNPLSGRRSFNRTPKGWISENIPIVITTEPTDDTTVPESEDL

SEQ ID NO: 3 - DNA, 1207 bp, Mus musculus
gagagccgaccaccaactgagcagctggtcagatccacctccaccatgccactctcaggaaccccggcccctaacaagaggaggaagtcaagcaaactgattatggagctcactggaggtggccgggagagctcaggcctgaacctgggcaagaagatcagtgtcccaagggatgtgatgttggaggagctgtcccttcttaccaaccgaggctccaagatgttcaagctacggcagatgcgggtggagaaatttatctatgagaatcaccccgatgttttctctgacagctcaatggatcacttccagaagtttcttcccacagtgggaggacagctggagacagctggtcagggcttctcatatggcaagggcagcagtggaggccaggctggcagcagtggctctgctggacagtatggctctgaccgtcatcagcagggctctgggtttggagctgggggttcaggtggtcctgggggccaggctggtggaggaggagctcctggcacagtagggcttggagagcccggatcaggtgaccaggcaggtggagatggaaaacatgtcactgtgttcaagacttatatttccccatgggatcgggccatgggggttgatcctcagcaaaaagtggaacttggcattgacctactggcatacggtgccaaagctgaactccccaaatataagtccttcaacaggacagcaatgccctacggtggatatgagaaggcctccaaacgcatgaccttccagatgcccaagtttgacctggggcctctgctgagtgaacccctggtcctctacaaccagaacctctccaacaggccttctttcaatcgaacccctattccctggttgagctctggggagcatgtagactacaacgtggatgttggtatccccttggatggagagacagaggagctgtgaagtgcctcctcctgtcatgtgcatcatttcccttctctggttccaatttgagagtggatgctggacaggatgccccaactgttaatccagtattcttgtggcaatggagggtaaagggtggggtccgttgcctttccacccttcaagttcctgctccgaagcatccctcctcaccagctcagagctcccatcctgctgtaccatatggaatctgctcttttatggaattttctctgccaccggtaacagtcaataaacttcaaggaaatga

SEQ ID NO: 4 - protein, 296 aa, Mus musculus
MPLSGTPAPNKRRKSSKLIMELTGGGRESSGLNLGKKISVPRDVMLEELSLLTNRGSKMFKLRQMRVEKFIYENHPDVFSDSSMDHFQKFLPTVGGQLETAGQGFSYGKGSSGGQAGSSGSAGQYGSDRHQQGSGFGAGGSGGPGGQAGGGGAPGTVGLGEPGSGDQAGGDGKHVTVFKTYISPWDRAMGVDPQQKVELGIDLLAYGAKAELPKYKSFNRTAMPYGGYEKASKRMTFQMPKFDLGPLLSEPLVLYNQNLSNRPSFNRTPIPWLSSGEHVDYNVDVGIPLDGETEEL

SEQ ID NO: 5 - DNA, 1261 bp, Homo sapiens
  modified_base (1221): n = a, c, g or t/u
cggtcacagcagctcagtcctccaaagctgctggaccccagggagagctgaccactgcccgagcagccggctgaatccacctccacaatgccgctctcaggaaccccggcccctaataagaagaggaaatccagcaagctgatcatggaactcactggaggtggacaggagagctcaggcttgaacctgggcaaaaagatcagtgtcccaagggatgtgatgttggaggaactgtcgctgcttaccaaccggggctccaagatgttcaaactgcggcagatgagggtggagaagtttatttatgagaaccaccctgatgttttctctgacagctcaatggatcacttccagaagttccttccaacagtggggggacagctgggcacagctggtcagggattctcatacagcaagagcaacggcagaggcggcagccaggcagggggcagtggctctgccggacagtatggctctgatcagcagcaccatctgggctctgggtctggagctgggggtacaggtggtcccgcgggccaggctggcaaaggaggagctgctggcacaacaggggttggtgagacaggatcaggagaccaggcaggcggagaaggaaaacatatcactgtgttcaagacctatatttccccatgggagcgagccatgggggttgacccccagcaaaaaatggaacttggcattgacctgctggcctatggggccaaagctgaacttcccaaatataagtccttcaacaggacggcaatgccctatggtggatatgagaaggcctccaaacgcatgaccttccagatgcccaagtttgacctggggcccttgctgagtgaacccctggtcctctacaaccaaaacctctccaacaggccttctttcaatcgaacccctattccctggctgagctctggggagcctgtagactacaacgtggatattggcatccccttggatggagaaacagaggagctgtgaggtgtttcctcctctgatttgcatcatttcccctctctggctccaatttggagagggaatgctgagcagatagcccccattgttaatccagtatccttatgggaatggagggaaaaaggagagatctacctttccatcctttactccaagtccccactccacgcatccttcctcaccaactcagagctccccttctacttgctccatatggaacctgctcgtttatggaatttntctgccaccagtaacagtcaataaacttcaaggaaaatga

SEQ ID NO: 6 - protein, 299 aa, Homo sapiens [sequence omitted]

SEQ ID NO: 7 - DNA, 982 bp, Mus musculus
attcggcacatgggatcgagggaccatgccgttccaggttcaaggataaaacccattgggccatagtgccgtcatattccaccttcagtgccttcctccacaattgggattcacccctgctgaaaagcgcacgctgacagcaagggaacaaaaaactatgctatcacatagtgccatggtgaagcaaaggaaacagcaagcatcagccatcacgaaggaaatccatggacatgatgttgacggcatggacctgggcaaaaaagttagcatccccagagacatcatgatagaagaattgtcccatttcagtaatcgtggggccaggctgtttaagatgcgtcaaagaagatctgacaaatacacctttgaaaatttccagtatgaatctagagcacaaattaatcacaatatcgccatgcagaatgggagagttgatggaagcaacctggaaggtggctcacagcaaggcccctcaactccgcccaacacccccgatccacgaagccccccaaatccagagaacatcgcaccaggatattctggaccactgaaggaaattcctcctgaaaggtttaacacgacggccgttcctaagtactaccggtctccatgggagcaggcgattggcagcgatccggagctcctggaggctttgtacccaaaacttttcaagcctgaaggaaaagcagaactgcgggattacaggagctttaacagggttgccactccatttggaggttttgaaaaagcatcaaaaatggtcaaattcaaagttccagattttgaactactgctgctgacagatcccaggttcttggcctttgccaatcctctttcgggcagacgatgctttaacagggcgccaaaggggtgggtatctgagaatatccccgtcgtgatcacaactgagcctacagaagacgccactgtaccggaatcagatgacctgtgagagggaagctggggatgccacaggaagttc

SEQ ID NO: 8 - protein, 264 aa, Mus musculus
MLSHSAMVKQRKQQASAITKEIHGHDVDGMDLGKKVSIPRDIMIEELSHFSNRGARLFKMRQRRSDKYTFENFQYESRAQINHNIAMQNGRVDGSNLEGGSQQGPSTPPNTPDPRSPPNPENIAPGYSGPLKEIPPERFNTTAVPKYYRSPWEQAIGSDPELLEALYPKLFKPEGKAELRDYRSFNRVATPFGGFEKASKMVKFKVPDFELLLLTDPRFLAFANPLSGRRCFNRAPKGWVSENIPVVITTEPTEDATVPESDDL

SEQ ID NO: 9 - DNA, 3330 bp, Homo sapiens
gggacgccacgcaactctcagcttcccgacagaggtgttaatcttgagggtctaagattccctcctgcctattgaggtcccatcctctcaggatgatccccaaggagcagaaggggccagtgatggctgccatgggggacctcactgaaccagtccctacgctggacctgggcaagaagctgagcgtgccccaggacctgatgatggaggagctgtcactacgcaacaacagagggtccctcctcttccagaagaggcagcgccgtgtgcagaagttcactttcgagttagcagccagccagcgggcgatgctggccggaagcgccaggaggaaggtgactggaacagcggagtcggggacggttgccaatgccaatggccctgaggggccgaactaccgctcggagctccacatcttcccggcctcacccggggcctcactcgggggtcccgagggcgcccaccctgcagccgcccctgctgggtgcgtccccagccccagcgccctggcgccaggctatgcggagccgctgaagggcgtcccgccagagaagttcaaccacaccgccatccccaagggctaccgctgcccttggcaggagttcgtcagctaccgggactaccagagcgatggccgaagtcacacccccagccccaacgactaccgaaatttcaacaagaccccggtgccatttggaggacccctcgtggggggcacttttcccaggccaggcacccccttcatcccggagcccctcagtggcttggaactcctccgtctcagacccagcttcaacagagtggcccagggctgggtccgtaacctcccagagtccgaggagctgtagccctagcctgaatcttcagttccccagtctcgggggcctggtaacatccggagccaagacttgtggacagcacttcacagttgaagaagggccttcacacacaaaacctgattgcaaatggcttcagaggtcaccaagttcagtcgtcccaaaacatgggtgtgtttcaaaattacctggggatgttgttccaaatccagacaactggactgtcccagacttgcagcatcagagtctcctgagtcgaggaatctgtattattaatagcaaccagggccgggtgtcgtggctcacgcctgtcatcccagcactttgggaggccgaggcaggaggatcacctgaggtcaggagttttgagaccagtctggccaaaatagtggaaccccgtcgctactaaaaatacaaaaatgagtcggacatggtggtgcatgcctgtaatcccagctacttgggaggctgagacaggagaatcacttgaactaggaggcagaggttgcagtgagccgagattgcgccactgcaccccagcctggacaacagagtgagactccttctcaaaagtaaataaataaatagcaaccagtactccaggtgattccagcataacttatccatggtttgtgtcattaggagtccacatccacacctctgctctttcctgttcctgtagtgtacactcccccggtgacagggtgctcactggcaccccatcttcctgtgaataactcaaataattagaaaatgttccttttactgagatgcagttggtcttcatctattcatgctctaaacagttcctaagcgctgactgtgcgctagacactgccaggcccgggcctcgaggaggaaaagacagtagggaagacattatagagcatgaagtcaccataattttccctaaagcatgcttattgacaattgaggaacaaagtgttgggagcagaagaaggagtccctcaccctaggtgtgagatgggattctggaagcttcctgaaggatttgagtgggaccttgtgggaggcgtgagagtccatgaagggggtgtgagggggagggtatttctggaaagtggaccagcatgtgcaaaaatatggaactgagcacgggtgcagggtgttctgcagaagggagaaggctgtgctagaggagccagtgagggccagcatggggtgggcttcactaaggaaatggggaaggttttagtgatgggtcttgctgggtgctgtgtggggcgcatattggagaagggtaatgccagaagccaggaagcctgcaagggatgaggccatgggaatggagagaaggggccacccactgggcacctaacaggacaggtgcaaagtggggtgcttattaagattccttctttccactccattttgagcaggctgcttaaagtggtggtgatgatgatgatgatgatggcagctttatatcgagtgcctcagtgcttgggctggtagtagtttctctacatatcttatttctaattctcagaacaaccctgagagaaagatattgttgtccccactttacagatgtggatatttaggccaaaaggaggaagtgactttccaggggcagacaccaaatgggaatctgattccagtggatgtctcttttcagtgcactgggtggtcaatgcccactcgctctgaaatcatctgactgtgatgccctgccttggagtttagaagttgagtgcaggcttgggagtcagactggatggggtaggttctaactctgccactgctagccggatgaacttgagcaagtcatttcacatctccgagcctctgtttctccaagtgtaagatgaggacaagtataaaacctcctttatgggtttgttgtgaacacagtgcagggcacatttataataagagctcagtcaatggtaggtttcatgcaactgctgctctaggctggaaaagttgttcttgcactggatgcagcatgagaagctggctgctaagatgtcactgggggtcactaaagctgaagcctgaaggaaagcctctcattgctgtagagctctccctgcctctctctctgggggcgatggggaaggtcaggagtccagcccattcccagggtgtgtgggatagcgattgcattttccttttgctctggagtttcactccccttctgggtcccaagggcccaatggcctgacttttagaattgcttgcaattggtgttttctcttgaatttgggggctgccatttaaagccaggtttccatgagctgaagaccagccattcaagaatctgaaaagtagacaagaggactccagttgcctcaggttggttctgctgtgctctggaaagtaactgcagccaccaggtatgaaaaggagcctggtggggagaccactgcacccaaaacaaatcctttcttcttctgagaatgtgactttttctggtgttgtaaaaaagaaaaaaaaaaagaatgctcattgtaaaaaaaaaaaaaaaaaa

SEQ ID NO: 10 - protein, 251 aa, Homo sapiens
MIPKEQKGPVMAAMGDLTEPVPTLDLGKKLSVPQDLMMEELSLRNNRGSLLFQKRQRRVQKFTFELAASQRAMLAGSARRKVTGTAESGTVANANGPEGPNYRSELHIFPASPGASLGGPEGAHPAAAPAGCVPSPSALAPGYAEPLKGVPPEKFNHTAIPKGYRCPWQEFVSYRDYQSDGRSHTPSPNDYRNFNKTPVPFGGPLVGGTFPRPGTPFIPEPLSGLELLRLRPSFNRVAQGWVRNLPESEEL

SEQ ID NO: 11 - DNA, 913 bp, Mus musculus
gtcggactgcaatagacacacaggccataaaactccagcttcccgactgaagtgttaatcttgggggtctgacatttcttcccatctactgtggccccaccaggatgatccccaaggagcagaaggagccagtgatggctgtcccgggggaccttgctgaaccagtcccttcgctggacctggggaagaagctgagcgtgcctcaggacctaatgatagaggagctgtctctacgaaacaaccgcggatccctcctctttcagaagaggcagcgccgggtgcagaagtttacctttgagctatcagaaagtttgcaggccatcctggcgagtagtgcccgagggaaagtggctggcagagcggcgcaggcaacggttcccaatggcttggaggagcagaaccaccactccgagacgcacgtgttccaggggtcacctggggaccccgggatcacccatctgggagcagcggggactgggtcggtccgtagtccaagcgccctggcaccaggctatgcagagcccctgaagggcgtcccaccggagaagttcaaccacactgccatccccaaaggctaccggtgcccttggcaggagttcaccagctaccaagactactcgagtggcagcagaagtcacactcccatcccccgagactatcgcaacttcaacaagacccccgtgccatttggaggaccccacgtgagggaggccattttccacgcaggcaccccctttgtcccggagtccttcagtggcttggaacttctccgcctcagacccaatttcaacagggttgctcagggctgggtccggaagctcccggagtctgaggaactgtagcctcagcctgaagctacaattccctgggctcaagaaacatgcttgtcttgaaaaaaaaaaaaaaaaaaaaa

SEQ ID NO: 12 - protein, 245 aa, Mus musculus
MIPKEQKEPVMAVPGDLAEPVPSLDLGKKLSVPQDLMIEELSLRNNRGSLLFQKRQRRVQKFTFELSESLQAILASSARGKVAGRAAQATVPNGLEEQNHHSETHVFQGSPGDPGITHLGAAGTGSVRSPSALAPGYAEPLKGVPPEKFNHTAIPKGYRCPWQEFTSYQDYSSGSRSHTPIPRDYRNFNKTPVPFGGPHVREAIFHAGTPFVPESFSGLELLRLRPNFNRVAQGWVRKLPESEEL